(19) United States
(12) Patent Application Publication
(10) Pub. No.: US 2026/0263365 A1
(43) Pub. Date: Sep. 10, 2026

(54) PRODUCTION AND USE OF EXTRACELLULAR VESICLE-CONTAINED ENAMPT

(71) Applicant: Washington University, St. Louis, MO (US)

(72) Inventors: Shin-Ichiro Imai, St. Louis, MO (US); Mitsukuni Yoshida, St. Louis, MO (US)

(73) Assignee: Washington University, St. Louis, MO (US)

(21) Appl. No.: 19/552,928

(22) Filed: Feb. 27, 2026

Related U.S. Application Data
(62) Division of application No. 17/617,245, filed on Jun. 8, 2020, now abandoned.
(60) Provisional application No. 62/858,483, filed on Jun. 7, 2019.
(60) Provisional application No. 62/988,232, filed on Mar. 11, 2020.

(51) Int. Cl. A61K 9/1271; A61K 9/1277; A61K 38/45; A61P 3/10; A61P 25/00; A61P 25/20

(52) U.S. Cl. CPC A61K 9/1271; A61K 9/1277; A61K 38/45; A61P 3/10; A61P 25/00; A61P 25/20; C12Y 204/02012

(57) ABSTRACT

The present invention relates to various compositions comprising NAMPT and/or mutant thereof, processes for preparing these compositions, and various methods of using these compositions to prevent or treat an age-associated condition in a subject. The present invention also relates to methods of increasing NMN and/or NAD+ biosynthesis in a subject or in a cell.

### CROSS-REFERENCE TO RELATED APPLICATIONS

This application is a divisional application of U.S. patent application Ser. No. 17/617,245 filed on Dec. 7, 2021, which is a Section 371 U.S. National Application of PCT/US2020/036616 filed on Jun. 8, 2020, which claims the benefit of U.S. Patent Application No. 62/988,232 filed on Mar. 11, 2020 and U.S. Patent Application No. 62/858,483 filed on Jun. 7, 2019, each of which is incorporated herein by reference in its entirety.

### STATEMENT REGARDING FEDERALLY SPONSORED RESEARCH OR DEVELOPMENT

This invention was made with government support under AG037457 and AG047902 awarded by the National Institutes of Health. The government has certain rights in the invention.

### SEQUENCE LISTING STATEMENT

A sequence listing containing the file named “019077USD1 Sequence Listing.xml” which is 9,222 bytes (measured in MS-Windows®) and created on Feb. 26, 2026, contains 6 nucleotide sequences, is provided herewith via the USPTO's EFS system, and is incorporated herein by reference in its entirety.

### FIELD OF THE INVENTION

The present invention relates to various compositions comprising NAMPT and/or mutant thereof, processes for preparing these compositions, and various methods of using these compositions to prevent or treat an age-associated condition in a subject. The present invention also relates to methods of increasing NMN and/or NAD+ biosynthesis in a subject or in a cell.

### BACKGROUND

Aging is a significant risk factor for impaired tissue functions and chronic diseases. In recent years, nicotinamide adenine dinucleotide (NAD+) metabolism has emerged as a central topic in the field of aging and longevity research due to an apparent age-associated decline in systemic NAD+ availability across many species (Canto et al., 2015; Rajman et al., 2018; Verdin, 2015; Yoshino et al., 2018). It has now been established that NAD+ availability declines over age at a systemic level, triggering a variety of age-associated pathophysiological changes in diverse model organisms. In mammals, the age-associated decline in NAD+ availability appears to be caused by two major events: decreased NAD+ biosynthesis and increased NAD+ consumption (Imai, 2016; Imai and Guarente, 2014). The former could be caused by chronic inflammation with enhanced oxidative stress and/or increased inflammatory cytokines, whereas the latter could be caused by increased DNA damage. As a consequence, NAD+ levels decrease with age in multiple tissues, including adipose tissue, skeletal muscle, liver, pancreas, skin, neurosensory retina, and brain (Canto et al., 2015; Lin et al., 2018; Rajman et al., 2018; Verdin, 2015; Yoshino et al., 2018). The realization of such systemic NAD+ decline as a fundamental event for age-associated pathophysiology has now provided a strong rationale to develop effective anti-aging interventions using key NAD+ intermediates, such as nicotinamide mononucleotide (NMN) and nicotinamide riboside (NR) (Rajman et al., 2018; Yoshino et al., 2018). Indeed, many studies have already proven the efficacy of NMN and NR to mitigate age-associated functional decline and treat age-associated disease conditions in various mouse models (Rajman et al., 2018; Yoshino et al., 2018).

In mammals, nicotinamide phosphoribosyltransferase (NAMPT) is the rate limiting enzyme in a major NAD+ biosynthetic pathway, converting nicotinamide and 5′-phosphoribosyl-pyrophosphate (PRPP) to NMN (Garten et al., 2015; Imai, 2009). Interestingly, there are two distinct forms of NAMPT in mammals: intra- and extracellular NAMPT (iNAMPT and eNAMPT, respectively) (Revollo et al., 2007). Whereas the function of iNAMPT as a critical NAD+ biosynthetic enzyme has been fully established, the physiological relevance and function of eNAMPT has long been controversial. eNAMPT was previously identified as pre-B cell colony-enhancing factor (PBEF) and insulin-mimetic visfatin, neither of which has been reconfirmed to date (Fukuhara et al., 2007; Garten et al., 2015; Imai, 2009; Samal et al., 1994). Additionally, eNAMPT was also reported to function as a proinflammatory cytokine, although this particular function has not yet been confirmed in loss- or gain-of-function Nampt mutants (Dahl et al., 2012). We have previously demonstrated the physiological relevance of eNAMPT in vivo by adipose tissue-specific genetic manipulation of Nampt (Yoon et al., 201 Yoon et al., entitled “SIRT1-Mediated eNAMPT Secretion from Adipose Tissue Regulates Hypothalamic NAD(+) and Function in Mice,” (2015) Cell Metab 21, 706-717. Adipose tissue-specific Nampt knockout (ANKO) mice, particularly females, show significant decreases in circulating eNAMPT levels. Surprisingly, ANKO mice exhibit a significant reduction in NAD+ levels not only in adipose tissue, but also in other remote tissues such as the hypothalamus (Yoon et al., 2015). Subsequent intensive investigations have revealed a novel function of eNAMPT that enhances NAD+, SIRT1 activity, and neural activation in the hypothalamus in response to fasting. These findings suggest the existence of a novel inter-tissue communication system between adipose tissue and the hypothalamus, mediated by eNAMPT (Imai, 2016).

However, how exactly eNAMPT regulates hypothalamic NAD+ levels has remained elusive. Moreover, there is currently no method for effectively delivering eNAMPT in vitro or in vivo or for applying it to mitigate any disease or condition.

### BRIEF SUMMARY

The present invention relates to various compositions comprising lipids and NAMPT and/or mutant thereof, processes for preparing these compositions, and various methods of using these compositions to prevent or treat an age-associated condition in a subject. The present invention also relates to methods of increasing NMN and/or NAD+ biosynthesis in subject or in a cell.

Other objects and features will be in part apparent and in part pointed out hereinafter.

### BRIEF DESCRIPTION OF THE DRAWINGS

FIG. 1A. Plasma eNAMPT levels of female and male mice at 6 and 18 months of age (n=5).

FIG. 1B. Plasma eNAMPT concentrations of female and male mice at 6 and 18 months of age over a 24-hr period (n=5 per time point per age). The differences between plasma eNAMPT levels at 6 and 18 months of age were assessed by two-way repeated measures ANOVA, and the age effects were significant in both males and females (p<0.05).

FIG. 1C. Plasma eNAMPT levels of mice and humans across different age groups (n=9 for mice; n=13 for humans).

FIG. 1D. Relationship of plasma eNAMPT levels and the remaining lifespans of individual mice (n=8). eNAMPT levels were measured at 26-28 months of age.

FIG. 2A. Plasma eNAMPT levels of control (left bars) and ANKI mice (right bars) at 4 months of age (n=3-4 per group per sex).

FIG. 2B. Plasma eNAMPT levels of control (left bars) and ANKI mice (right bars) at 24 months of age (n=3-4 per group per sex).

FIG. 2C. Tissue NAD+ levels of control and ANKI mice at 20 months of age (n=3 per group per sex).

FIG. 3A. Wheel-running activity of control (CTRL) and ANKI female mice at 4 and 18 months of age (4-month old, n=3; 18-month old, n=10-13 per group).

FIG. 3B. Total ambulatory (upper panel) and rearing (lower panel) activities of control and ANKI female mice at 18 months of age (n=4-5 per group).

FIG. 3C. The levels of sleep fragmentation in 4 and 20 month-old male mice (n=6 per group) and control (left bar) and ANKI mice (right bar) at 20 months of age (n=7-8 per group; male and female mice combined). The numbers of transitions between NREM sleep (NR) and wake (W) cycles are shown.

FIG. 3D. mRNA expression levels of Ox2r and Prdm13 in the hypothalamus of control and ANKI female mice at 20 months of age (n=3-6 per group).

FIG. 4A. Blood glucose (n=8-13) and insulin (n=8-9).

FIG. 4B. Blood glucose (n=8-13) levels during the IPGTTs in control (left bars) and ANKI male mice (right bars) at 17-20 months of age.

FIG. 4C. Total numbers of pancreatic islets in the pancreata of control (left bar) and ANKI male mice (right bar) at 20 months of age (n=3 per group).

FIG. 4D. Representative images of pancreatic islets in the pancreata of control and ANKI male mice at 20 months of age (n=3 per group).

FIG. 4E. Size distributions of pancreatic islets in the pancreata of control (left bars) and ANKI male mice (right bars) at 20 months of age (n=3 per group).

FIG. 4F. Scotopic a, scotopic b, and photopic b waves from ERG analysis of control and ANKI mice at 18-20 months of age (n=6-7 per group; male and female combined).

FIG. 5A. Kaplan-Meier curves of female and male ANKI mice (females, control 39, ANKI 40; males, control 39, ANKI 39).

FIG. 5B. Age-associated mortality rates of control and ANKI female and male mice.

FIG. 6A. Comparison of eNAMPT, extracellular vesicle (EV) marker proteins (TSG101, CD63, CD81, and CD9), and non-EV proteins (transferrin and albumin) in whole plasma, EV fraction, and supernatant isolated by ultracentrifugation and the Total Exosome Isolation (TEI) kit. The protein concentrations were typically ~0.4 and ~1 μg/μl for EVs purified by ultracentrifugation and the TEI kit, respectively, when EVs were reconstituted with an equal volume of PBS to the starting volume of plasma. 40 μg of protein from each fraction were loaded.

FIG. 6B. Comparison of eNAMPT and EV marker proteins in six fractions (F1-6) isolated from sucrose density-gradient centrifugation. 2 ml of plasma were used for this fractionation.

FIG. 6C. Comparison of eNAMPT in whole plasma (P), EV fraction (E), and supernatant(S) isolated from three 4 month-old male mice and 37, 41, and 45 year-old male human donors. Each fraction was loaded after adjusting them to an equal volume.

FIG. 6D. Comparison of eNAMPT, TSG101, transferrin, and immunoglobulin light chain (Ig LC) in the treatment of mouse plasma with proteinase K and/or Triton X-100.

FIG. 7A. Fluorescent images of primary hypothalamic neurons following the incubation with BODIPY-labeled EVs. EVs purified from 400 μl of mouse plasma were added to 200 μl of culture media. Arrowheads indicate neurons that internalized BODIPY-labeled EVs.

FIG. 7B. Cytoplasmic levels of FLAG-tagged recombinant NAMPT (recNAMPT) and cellular NAD+ levels in the primary hypothalamic neurons after incubated with recNAMPT alone or EV-contained recNAMPT (n=3).

FIG. 7C. Relative rate of NAD+ biosynthesis in primary hypothalamic neurons after incubating with EVs isolated from plasma with ultracentrifugation and TEI kit (n=4).

FIG. 7D. Relative cellular NAMPT activity in primary hypothalamic neurons after incubating with control (CTRL) and Nampt-knockdown (NAMPT-KD) EVs generated from OP9 adipocytes (n=3-6).

FIG. 7E. Levels of cytoplasmic NAMPT and NAD+ in primary hypothalamic neurons after incubated with EVs isolated from 6 and 18 month-old mice (n=4).

FIG. 7F. NAD+ level changes after incubating with EVs isolated from 6 and 20-22 month-old mice. (n=9-13).

FIG. 7G. NAD+ levels in primary hypothalamic neurons after incubating with EVs isolated from control (left bars) and ANKI mice (right bars) at 20 months of age.

FIG. 7H. Total wheel-running activity counts of 20 month-old female mice during dark and light times before and after 4 consecutive daily injections of EVs purified from 4-6 month-old mice (n=5).

FIG. 7I. Total wheel-running activity counts of 25 month-old female mice during the dark time before and after 4 consecutive daily injections of control (CTRL) and Nampt-knockdown (NAMPT-KD) EVs purified from OP9 adipocytes (n=6).

FIG. 7J. Kaplan-Meier curves and representative images.

FIG. 7K. Kaplan-Meier curves of aged female mice injected with vehicle or EVs isolated from 4-12 month-old mice (n=11-12). The mouse images were taken after 3 months of treatment.

FIG. 8A. NAMPT levels in primary adipocytes isolated from 6 and 18 month-old male mice (n=5).

FIG. 8B. Western blots for plasma eNAMPT levels over the course of 24 hrs in female mice at 6 and 18 months of age (n=5 per time point per age). 6-1~5 and 18-1~5 are individual plasma samples at 6 and 18 months of age, respectively. The order of samples in each blot was intentionally randomized to avoid biases in signal quantitation.

FIG. 8C. Western blots for plasma eNAMPT levels over the course of 24 hrs in male mice at 6 and 18 months of age (n=5 per time point per age). 6-1~5 and 18-1~5 are individual plasma samples at 6 and 18 months of age, respectively. The order of samples in each blot was intentionally randomized to avoid biases in signal quantitation.

FIG. 9A. Plasma eNAMPT levels of 6 month-old control, 18 month-old ANKI, and 18 month-old control mice (n=3). Plasma eNAMPT shows doublet bands (right panel), both of which were quantitated (left panel).

FIG. 9B. Relationship of plasma eNAMPT levels and hypothalamic NAD+ levels in control and ANKI female mice at 20 months of age (n=5 per group).

FIG. 10A. Wheel-running activity of 6 and 18 month-old wild-type mice (n=3-9). Differences were assessed by Wilcoxon matched-pairs singled-ranked test.

FIG. 10B. Ambulatory (top) and rearing (bottom) activities of control (CTRL) and ANKI male mice at 18 months of age (n=4-7).

FIG. 11A. Relative blood glucose levels of control and ANKI male mice (right bars) at 17-20 months of age during insulin tolerance tests (n=8 per group). Glucose levels at each time point are normalized to that at 0 min time point.

FIG. 11B. Body weights of control (left bars) and ANKI female and male mice (right bars) at the age of 18-20 months of age (male, n=20-36; female, n=19-23).

FIG. 11C. Body compositions of control (left bars) and ANKI male and female mice (right bars) at 17-20 months of age.

FIG. 11D. Daily food intakes of control (left bars) and ANKI male and female mice (right bars) at 23 months of age (n=6-9).

FIG. 11E. Relative plasma cytokine levels of control (left bars) and ANKI male and female mice (right bars) at 23 months of age (n=3).

FIG. 11F. Contextual fear conditioning test of control and ANKI female mice at 20 months of age. First graph: Percent freezing time of control and ANKI mice at baseline and during shock-tone training; Second graph: Contextual fear response on day 2; Third graph: Baseline and auditory cue response on day 3 (n=5).

FIG. 12A. eNAMPT and EV marker proteins in six fractions isolated by flotation of ultracentrifugation purified EVs into a sucrose-density gradient.

FIG. 12B. Densities of 12 fractions and the comparison of eNAMPT and an EV marker Alix in each fraction isolated from a sucrose-density gradient separation of EVs purified by the Total Exosome Isolation (TEI) kit. A very minor fractions of eNAMPT and Alix, which were cofractionated in Fraction #11, are most likely due to a contamination of protein aggregates.

FIG. 12C. Electron microscopic images and particle size distributions of EVs isolated from mouse and human plasma.

FIG. 12D. Electron microscopic images and particle size distributions of EVs isolated from mouse (n=100 for each analysis).

FIG. 12E. Comparison of eNAMPT, EV marker proteins (CD9, C81, Hsp70, and TSG101), and non-EV proteins (adiponectin and adipsin) in the conditioned media of OP9 adipocytes. The EV fraction and supernatant were separated by ultracentrifugation. 40 μg of protein from each fraction was loaded.

FIG. 13A. Fluorescent images of primary hypothalamic neurons following the incubation with BODIPY labeled EVs from OP9 adipocytes.

FIG. 13B. Relative NAMPT enzymatic activity in primary hypothalamic neurons after incubating with each fractions (Media, EV, and supernatant) isolated from the conditioned media of OP9 adipocytes. Each NMN biosynthesis level measured by mass spectrometry with D-4-NAM was normalized to that in untreated cells.

FIG. 13C. eNAMPT levels in EVs isolated from control and Nampt-knockdown (NAMPT-KD) OP9 adipocytes. Protein concentrations of EVs purified from both conditioned media were very similar, suggesting that there was no difference in the amounts of EVs released from both cell lines.

FIG. 13D. Levels of cytoplasmic NAMPT in primary hypothalamic neurons after incubated with plasma from 6 and 18 month-old mice.

FIG. 13E. Total wheel-running activity counts throughout 24 hrs of 20 month-old female mice before and after four consecutive daily injections of EVs isolated from 4-6-month old mice (n=5).

FIG. 13F. Total wheel-running activity counts of 25 month-old female mice during the light time before and after 4 consecutive daily injections of EVs purified from control (CTRL) and NAMPT-KD OP9 adipocytes (n=5-6).

FIG. 13G. Total wheel-running activity counts of 20 month-old male mice during dark and light times before and after 4 consecutive daily injections of EVs purified from 4-6-month old mice (n=5).

FIG. 14. Cultured primary mouse hippocampal neurons treated with mouse plasma-derived EVs (****p<0.0001 by one-way ANOVA with Sidak correction for multiple comparisons).

FIG. 15. Astrocyte-enriched cultures treated with Bodipy-TR-Ceramide labeled EVs.

FIG. 16. Microglia cultures treated with Bodipy-TR-Ceramide labeled EVs.

### DETAILED DESCRIPTION

The present invention relates to various compositions comprising nicotinamide phosphoribosyltransferase (NAMPT) and/or mutant thereof, processes for preparing these compositions, and various methods of using these compositions to prevent or treat an age-associated condition in a subject. The present invention also relates to methods of increasing NMN and/or NAD+ biosynthesis in a cell. The methods and compositions allow for an improved delivery system of NAMPT and/or mutant thereof to cells and organisms where it can be utilized and act as an anti-aging modifier.

The present invention is based on the discovery that circulating levels of extracellular nicotinamide phosphoribosyltransferase (eNAMPT) significantly decline with age in mice and humans. Increasing circulating eNAMPT levels in aged mice by adipose-tissue specific overexpression of NAMPT increases NAD+ levels in multiple tissues, thereby enhancing their functions and extending healthspan in female mice. However, prior to the instant invention, it was unclear how eNAMPT is delivered in vivo or how levels of NAMPT could be increased in aging individuals.

It has been discovered that extracellular vesicle (EV) delivery of NAMPT provides an effective method of supplementing NAMPT in a cell system or individual. eNAMPT is carried in extracellular vesicles (EVs) through systemic circulation in mice and humans. Delivery of eNAMPT via EV results in cellular internalization and NAD+ biosynthesis. Supplementing eNAMPT-containing EVs isolated from young mice significantly improves wheel-running activity and extends lifespan in aged mice. Thus, the inventors have revealed a novel EV-mediated delivery mechanism for eNAMPT, which promotes systemic NAD+ biosynthesis and counteracts aging, suggesting a potential avenue for anti-aging intervention in humans.

In recent years, many studies have reported an important role of EVs as a new inter-cellular or inter-tissue communication tool for transporting proteins and microRNAs (Whitham et al., 2018; Ying et al., 2017; Zhang et al., 2017). Indeed, it has recently been demonstrated that adipose tissue is a major source of circulating EV-contained microRNAs that regulate gene expression in distant tissues (Thomou et al., 2017). In this context, it is intriguing that eNAMPT in blood circulation is contained almost exclusively in EVs. In adipose tissue, SIRT1-dependent deacetylation of lysine 53 on iNAMPT predisposes the protein to secretion (Yoon et al., 2015), implicating that this deacetylation might be involved in the process of incorporating the NAMPT protein into EVs. However, how eNAMPT-containing EVs are targeted specifically to certain tissues, such as the hypothalamus, hippocampus, pancreas, and retina, remains unknown. It has been found that EV-mediated delivery is critical for eNAMPT to be properly internalized into cells and enhance NMN/NAD+ biosynthesis intracellularly. When giving the eNAMPT protein alone, the protein is not internalized properly.

Further, it has been found that eNAMPT-containing EVs can be transferred from one individual to another. In particular, it has been discovered that supplementing eNAMPT-containing EVs purified from young mice significantly enhances the wheel-running activity and extends lifespan in aged mice. In human blood, eNAMPT is also contained exclusively in EVs. Thus, this model supports the use of EV-contained eNAMPT as an anti-aging biologic in humans. These findings open a new possibility to use the EV-mediated systemic delivery of eNAMPT as a biologic for an effective anti-aging intervention.

Accordingly, various compositions of the present invention comprise nicotinamide phosphoribosyltransferase (NAMPT) and/or mutant thereof and lipids, wherein the lipids form a layer that at least partially encapsulates the NAMPT or mutant thereof. For example, the lipids can aggregate to form micelles or liposomes which encapsulates the NAMPT and/or mutant thereof. In some embodiments, the composition comprises exosomes comprising NAMPT and/or mutant thereof. In some embodiments, the composition comprises exosomes comprising NAMPT and/or mutant thereof.

In some embodiments, the lipid comprises a phospholipid. For example, the phospholipid can be selected from the group consisting of phosphatidic acid, phosphatidylethanolamine, phosphatidylcholine, phosphatidylserine, phosphatidylinositol, phosphatidylinositol phosphate, phosphatidylinositol bisphosphate, phosphatidylinositol trisphosphate, diphosphatidyl glycerol, and combinations thereof. In various embodiments, the lipid comprises a sphingolipid. For example, the sphingolipid can be selected from the group consisting of ceramide phosphorylcholine, ceramide phosphorylethanolamine, ceramide phosphoryl lipid, and combinations thereof.

In various embodiments, the concentration of NAMPT and/or mutant thereof in the composition is from about 1 wt. % to about 20 wt. %. In some embodiments, the composition has a weight ratio of NAMPT and/or mutant thereof that is from about 1:1 to about 100:1.

Typically, the composition comprises a plurality of vesicles. In various embodiments, the vesicles are characterized as having a mean particle size of from about 10 nm to about 200 nm, from about 10 nm to about 100 nm, or from about 20 nm to about 100 nm. In some embodiments, the vesicle further comprises water.

In various embodiments, the composition is free or essentially free (e.g., less than 1 wt. % or even less than 0.1 wt. %) of certain biological components. For example in some embodiments, the composition is free or essentially free (e.g., less than 1 wt. % or even less than 0.1 wt. %) of adipocytes, blood and/or blood plasma.

The compositions as described herein can be administered by a routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. In various embodiments, administration is selected from the group consisting of oral, intranasal, intraperitoneal, intravenous, intramuscular, rectal, and transdermal. In some embodiments, the composition may be administered orally. In various embodiments, the composition is administered parenterally.

A composition for oral administration can be formulated using pharmaceutically acceptable carriers and excipients known in the art in dosages suitable for oral administration. Such carriers enable the composition to be formulated as tablets, pills, dragees, capsules, liquids, gels, syrups, slurries, suspensions, and the like, for ingestion by the subject. In certain embodiments, the composition is formulated for parenteral administration. Further details on techniques for formulation and administration can be found in the latest edition of REMINGTON'S PHARMACEUTICAL SCIENCES (Mack Publishing Co., Easton, Pa., which is incorporated herein by reference). After compositions have been prepared, they can be placed in an appropriate container and labeled for treatment of an indicated condition. Such labeling would include amount, frequency, and method of administration.

In addition to the active ingredients (e.g., the inhibitor compound), the composition can contain suitable pharmaceutically acceptable carriers and excipients. In some embodiments, the composition further comprises a carrier. Carriers include, for example, water. Also, in various embodiments, the composition further comprises an excipient. Various excipients include, for example, various non-toxic, inert solid, semi-solid or liquid filler, diluent, encapsulating material, or formulation auxiliary of any type. Some examples of materials which can serve as pharmaceutically acceptable excipients are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil; and soybean oil; glycols such as propylene glycol; esters such as ethyl oleate and ethyl laurate; agar; detergents such as TWEEN 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring, and perfuming agents, preservatives and antioxidants can also be present in the composition.

As noted, the present invention also relates to various methods of using the NAMPT-containing compositions or NAMPT mutant-containing compositions described herein. One method is directed to increasing NMN and/or NAD+ biosynthesis in a cell. The method comprises applying the composition as described herein to the cell. Without being bound by theory, it is believed that the lipid membrane of the vesicles of the composition may fuse with the plasma membrane of the cell, thus facilitating the transfer of the vesicular contents (i.e., NAMPT) into the cell. Once internalized, NAMPT and/or mutant thereof may be used in cellular biosynthetic pathways to produce, for example, NMN and/or NAD+. Other methods include increasing NMN and/or NAD+ biosynthesis in a subject. These methods comprise administering to the subject a composition as described herein.

Another method is directed to preventing or treating an age-associated condition in a subject (e.g., a subject in need thereof). The method comprise administering to the subject an effective amount of the composition as described herein to the subject. The methods described herein can increase NMN and/or NAD+ biosynthesis above physiological levels. Physiological levels correspond to the amount of a product expected to be produced by a cell or an organism at a certain time. Production may vary naturally over the lifetime of an organism. Therefore, in various embodiments, an increase in NMN and/or NAD+ may be determined relative to the amount reasonably synthesized by the subject at that point in time.

In various embodiments, the age-associated condition comprises a physiological condition selected from the group consisting of: a decline in physical activity, a decline in sleep quality, a decline in cognitive function, a decline in glucose metabolism, a decline in vision and combinations thereof. In various embodiments, methods disclosed herein can be used for treating, ameliorating, mitigating, or reversing any age-associated disease or condition which involves NMN metabolism, such as, without limitation, type II diabetes, obesity, age-associated obesity, age-associated increases in blood lipid levels, age-associated decreases in insulin sensitivity, age-associated loss or decrease in memory function, age-associated loss or decrease in eye function, age-associated physiological decline, impairment in glucose-stimulated insulin secretion, diabetes, amelioration of mitochondrial function, neural death, and/or cognitive function in Alzheimer's disease, protection of heart from ischemia/reperfusion injury, maintenance of neural stem/progenitor cell populations, restoration of skeletal muscle mitochondrial function and arterial function following injury, and age-associated functional decline.

In various embodiments, the age-associated condition can comprise an age-associated loss of insulin sensitivity and/or insulin secretion in a subject in need thereof. In some embodiments, the age-associated condition comprises age-associated impairment of memory function. In various embodiments, the age-associated condition comprises a decline in eye function. In some embodiments, the decline in eye function includes age-associated retinal degeneration.

In some embodiments, age-associated condition can comprise a muscle disease and the present invention comprises methods of treating said muscle disease in a subject in need thereof. In various configurations, a muscle disease which can be treated in accordance with the present teachings includes, without limitation, muscle frailty, muscle atrophy, muscle wasting a decrease in muscle strength. In various configurations, a muscle disease which can be treated in accordance with the present teachings includes, without limitation, sarcopenia, dynapenia, cachexia, muscular dystrophy, myotonic disorders, spinal muscular atrophies, and myopathy. The muscular dystrophy can be, for example, Duchenne Muscular Dystrophy, Becker Muscular Dystrophy, Congenital Muscular Dystrophy, Distal Muscular Dystrophy, Emery-Dreifuss Muscular Dystrophy, Facioscapulohumeral Muscular Dystrophy, Limb-Girdle Muscular Dystrophy, or Oculopharyngeal Muscular Dystrophy. In some configurations, the myotonic disorder can be Myotonic Dystrophy, Myotonia Congenita, or Paramyotonia Congenita. In some configurations, the myopathy can be Bethlem myopathy, congenital fibre type disproportion, fibrodysplasia ossificans progressiva, hyper thyroid myopathy, hypo thyroid myopathy, minicore myopathy, multicore myopathy, myotubular myopathy, nemaline myopathy, periodic paralysis, hypokalemic myopathy or hyperkalemic myopathy. In some configurations, the muscle disease can be Acid Maltase Deficiency, Carnitine Deficiency, Carnitine Palmityl Transferase Deficiency, Debrancher Enzyme Deficiency, Lactate Dehydrogenase Deficiency, Mitochondrial Myopathy, Myoadenylate Deaminase Deficiency, Phosphorylase Deficiency, Phosphofructokinase Deficiency, or Phosphoglycerate Kinase Deficiency. In some configurations, the muscle disease can be sarcopenia, dynapenia or cachexia. In some configurations, the muscle disease can be sarcopenia.

Embodiments of preventing and treating an age-associated condition can include preventing age-associated functional decline in a subject in need thereof. In various configurations, the age-associated functional decline can result from or can be associated with, in non-limiting example, loss of appetite, low glucose levels, muscle weakness, malnutrition, or anorexia of aging. Other, non-limiting age-associated conditions that may be treated by the compositions described herein can include diabetes (e.g., Type II diabetes) and obesity.

In various embodiments, the present invention comprises administering the compositions described herein to facilitate the production of NMN/NAD+ in the subject.

A therapeutically effective dose refers to an amount of active ingredient which provides the desired result. The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. In some embodiments, the composition is administered at a dose providing from about 10 mg to about 500 mg, or about 50 to about 500 mg of NAMPT and/or mutant thereof per day to the subject. In some embodiments, the subject is human. In various embodiments, the subject is a human.

The present invention is also directed to a process for preparing various compositions described herein. In various embodiments, the method comprises separating the vesicle from a medium comprising a component selected from the group consisting of a culture containing adipocytes, blood and blood plasma.

In various embodiments, the medium is a culture comprising adipocytes. The adipocytes may overexpress a gene that codes for NAMPT (e.g., Nampt) or a biosynthetic precursor.

In various embodiments, the medium is a culture media comprising blood or blood plasma.

In various embodiments, the separation process comprises centrifugation (e.g., ultracentrifugation). In some embodiments, the separation process comprises an exosome isolation technique.

In various embodiments, the vesicles of the compositions described herein are synthetically or semi-synthetically derived. For example, the NAMPT and/or mutant thereof can be produced by recombinant techniques. Subsequently, the NAMPT and/or mutant thereof and lipids can be combined, for example, in an aqueous solvent.

### NAMPT and Mutants of NAMPT

In various embodiments, the composition comprises NAMPT. In some embodiments, the NAMPT comprises wild-type NAMPT of SEQ ID NO: 1. In some embodiments, the NAMPT comprises wild-type NAMPT of SEQ ID NO: 2.

| Source Organism | Amino Acid Sequence |
|---|---|
| Mouse | MNAAAEAEFNILLATDSYKVTHYKQYPPNTSKVYSYFECRE |
| (SEQ ID NO: 1) | KKTENSKVRKVKYEETVFYGLQYILNKYLKGKVVTKEKIQE |
| | AKEVYREHFQDDVFNERGWNYILEKYDGHLPIEVKAVPEGS |
| | VIPRGNVLFTVENTDPECYWLTNWIETILVQSWYPITVATNS |
| | REQKKILAKYLLETSGNLDGLEYKLHDFGYRGVSSQETAGIG |
| | ASAHLVNFKGTDTVAGIALIKKYYGTKDPVPGYSVPAAEHS |
| | TITAWGKDHEKDAFEHIVTQFSSVPVSVVSDSYDIYNACEKI |
| | WGEDLRHLIVSRSTEAPLIIRPDSGNPLDTVLKVLDILGKKFP |
| | VTENSKGYKLLPPYLRVIQGDGVDINTLQEIVEGMKQKKWSI |
| | ENVSFGSGGALLQKLTRDLLNCSFKCSYVVTNGLGVNVFKD |
| | PVADPNKRSKKGRLSLHRTPAGNFVTLEEGKGDLEEYGHDL |
| | LHTVFKNGKVTKSYSFDEVRKNAQLNIEQDVAPH |
| Human | MNPAAEAEFNILLATDSYKVTHYKQYPPNTSKVYSYFECRE |
| (SEQ ID NO: 2) | KKTENSKLRKVKYEETVFYGLQYILNKYLKGKVVTKEKIQE |
| | AKDVYKEHFQDDVFNEKGWNYILEKYDGHLPIEIKAVPEGF |
| | VIPRGNVLFTVENTDPECYWLTNWIETILVQSWYPITVATNS |
| | REQKKILAKYLLETSGNLDGLEYKLHDFGYRGVSSQETAGIG |
| | ASAHLVNFKGTDTVAGLALIKKYYGTKDPVPGYSVPAAEHS |
| | TITAWGKDHEKDAFEHIVTQFSSVPVSVVSDSYDIYNACEKI |
| | WGEDLRHLIVSRSTQAPLIIRPDSGNPLDTVLKVLEILGKKFP |
| | VTENSKGYKLLPPYLRVIQGDGVDINTLQEIVEGMKQKMWS |
| | IENIAFGSGGGLLQKLTRDLLNCSFKCSYVVTNGLGINVFKDP |
| | VADPNKRSKKGRLSLHRTPAGNFVTLEEGKGDLEEYGQDLL |
| | HTVFKNGKVTKSYSFDEIRKNAQLNIELEAAHH |

In various embodiments, the composition comprises a mutant of NAMPT. For example, two single amino acid mutants of the NAMPT protein, K53R and K53Q have been reported. K53 is acetylated on iNAMPT, and SIRT1 deacetylates this lysine, predisposing NAMPT to secretion. The K53R mutant is secreted ~3-fold higher than the wild-type NAMPT protein, whereas the K53Q mutant shows a significant decrease in secretion. Because K53R does not change the enzymatic activity of NAMPT, K53R mutant can exhibit a better efficiency to be packaged into exosomes and delivered to target tissues.

Accordingly, the mutant of NAMPT can comprise an amino acid sequence having an arginine or glutamine residue (particularly an arginine residue) at a position corresponding to position 53 of wild-type NAMPT of SEQ ID NO: 1 and wherein the remaining amino acid sequence of the mutant comprises at least 80%, at least 85%, at least 90%, at least 95%, at least 99%, at least 99.9%, or at least 99.99% sequence identity to SEQ ID NO: 1. In some embodiments, the mutant of NAMPT comprises an amino acid sequence having an arginine or glutamine residue (particularly an arginine residue) at a position corresponding to position 53 of wild-type NAMPT of SEQ ID NO: 2 and wherein the remaining amino acid sequence of the mutant comprises at least 80%, at least 85%, at least 90%, at least 95%, at least 99%, at least 99.9%, or at least 99.99% sequence identity to SEQ ID NO: 2.

In various embodiments, the mutant of NAMPT comprises at least 80%, at least 85%, at least 90%, at least 95%, at least 99%, at least 99.9%, or at least 99.99% sequence identity to the wild-type NAMPT of SEQ ID NO: 1 or SEQ ID NO: 2 and further comprises at least one amino acid substitution that removes an acetylation site as compared to the wild-type NAMPT. In some embodiments, the mutant of NAMPT is secreted from a cell more efficiently than the wild-type NAMPT or is packaged into an exosome more efficiently than the wild-type NAMPT.

The mutant of NAMPT can also include those having the following sequences:

| Source Organism | Amino Acid Sequence |
|---|---|
| Mouse Mutant - K53R | MNAAAEAEFNILLATDSYKVTHYKQYPPNTSKVYSYFECRE |
| (SEQ ID NO: 3) | KKTENSKVRKVRYEETVFYGLQYILNKYLKGKVVTKEKIQE |
| | AKEVYREHFQDDVFNERGWNYILEKYDGHLPIEVKAVPEGS |
| | VIPRGNVLFTVENTDPECYWLTNWIETILVQSWYPITVATNS |
| | REQKKILAKYLLETSGNLDGLEYKLHDFGYRGVSSQETAGIG |
| | ASAHLVNFKGTDTVAGIALIKKYYGTKDPVPGYSVPAAEHS |
| | TITAWGKDHEKDAFEHIVTQFSSVPVSVVSDSYDIYNACEKI |
| | WGEDLRHLIVSRSTEAPLIIRPDSGNPLDTVLKVLDILGKKFP |
| | VTENSKGYKLLPPYLRVIQGDGVDINTLQEIVEGMKQKKWSI |
| | ENVSFGSGGALLQKLTRDLLNCSFKCSYVVTNGLGVNVFKD |
| | PVADPNKRSKKGRLSLHRTPAGNFVTLEEGKGDLEEYGHDL |
| | LHTVFKNGKVTKSYSFDEVRKNAQLNIEQDVAPH |
| Human Mutant - K53R | MNPAAEAEFNILLATDSYKVTHYKQYPPNTSKVYSYFECRE |
| (SEQ ID NO: 4) | KKTENSKLRKVRYEETVFYGLQYILNKYLKGKVVTKEKIQE |
| | AKDVYKEHFQDDVFNEKGWNYILEKYDGHLPIEIKAVPEGF |
| | VIPRGNVLFTVENTDPECYWLTNWIETILVQSWYPITVATNS |
| | REQKKILAKYLLETSGNLDGLEYKLHDFGYRGVSSQETAGIG |
| | ASAHLVNFKGTDTVAGLALIKKYYGTKDPVPGYSVPAAEHS |
| | TITAWGKDHEKDAFEHIVTQFSSVPVSVVSDSYDIYNACEKI |
| | WGEDLRHLIVSRSTQAPLIIRPDSGNPLDTVLKVLEILGKKFP |
| | VTENSKGYKLLPPYLRVIQGDGVDINTLQEIVEGMKQKMWS |
| | IENIAFGSGGGLLQKLTRDLLNCSFKCSYVVTNGLGINVFKDP |
| | VADPNKRSKKGRLSLHRTPAGNFVTLEEGKGDLEEYGQDLL |
| | HTVFKNGKVTKSYSFDEIRKNAQLNIELEAAHH |
| Mouse Mutant - K53Q | MNAAAEAEFNILLATDSYKVTHYKQYPPNTSKVYSYFECRE |
| (SEQ ID NO: 5) | KKTENSKVRKVQYEETVFYGLQYILNKYLKGKVVTKEKIQE |
| | AKEVYREHFQDDVFNERGWNYILEKYDGHLPIEVKAVPEGS |
| | VIPRGNVLFTVENTDPECYWLTNWIETILVQSWYPITVATNS |
| | REQKKILAKYLLETSGNLDGLEYKLHDFGYRGVSSQETAGIG |
| | ASAHLVNFKGTDTVAGIALIKKYYGTKDPVPGYSVPAAEHS |
| | TITAWGKDHEKDAFEHIVTQFSSVPVSVVSDSYDIYNACEKI |
| | WGEDLRHLIVSRSTEAPLIIRPDSGNPLDTVLKVLDILGKKFP |
| | VTENSKGYKLLPPYLRVIQGDGVDINTLQEIVEGMKQKKWSI |
| | ENVSFGSGGALLQKLTRDLLNCSFKCSYVVTNGLGVNVFKD |
| | PVADPNKRSKKGRLSLHRTPAGNFVTLEEGKGDLEEYGHDL |
| | LHTVFKNGKVTKSYSFDEVRKNAQLNIEQDVAPH |
| Human Mutant -K53Q | MNPAAEAEFNILLATDSYKVTHYKQYPPNTSKVYSYFECRE |
| (SEQ ID NO: 6) | KKTENSKLRKVQYEETVFYGLQYILNKYLKGKVVTKEKIQE |
| | AKDVYKEHFQDDVFNEKGWNYILEKYDGHLPIEIKAVPEGF |
| | VIPRGNVLFTVENTDPECYWLTNWIETILVQSWYPITVATNS |
| | REQKKILAKYLLETSGNLDGLEYKLHDFGYRGVSSQETAGIG |
| | ASAHLVNFKGTDTVAGLALIKKYYGTKDPVPGYSVPAAEHS |
| | TITAWGKDHEKDAFEHIVTQFSSVPVSVVSDSYDIYNACEKI |
| | WGEDLRHLIVSRSTQAPLIIRPDSGNPLDTVLKVLEILGKKFP |
| | VTENSKGYKLLPPYLRVIQGDGVDINTLQEIVEGMKQKMWS |
| | IENIAFGSGGGLLQKLTRDLLNCSFKCSYVVTNGLGINVFKDP |
| | VADPNKRSKKGRLSLHRTPAGNFVTLEEGKGDLEEYGQDLL |
| | HTVFKNGKVTKSYSFDEIRKNAQLNIELEAAHH |

Having described the invention in detail, it will be apparent that modifications and variations are possible without departing from the scope of the invention defined in the appended claims.

### EXAMPLES

The following non-limiting examples are provided to further illustrate the present invention.

### Materials and Methods

The following materials (Table 1) and methods were used to perform the experiments in the following examples.

TABLE 1
Materials/Resources used in Examples

| | | |
|---|---|---|
| REAGENT or RESOURCE | SOURCE | IDENTIFIER |

Antibodies

| | | |
|---|---|---|
| NAMPT polyclonal antibody | Bethyl | A300-372A |
| NAMPT monoclonal antibody | Adipogen | OMNI379 |
| GAPDH | Millipore | MAB374 |
| CD63 | Santa Cruz | sc-5275 |
| Alix | Santa Cruz | Sc-53540 |
| CD81 | Santa Cruz | Sc-166029 |
| CD9 | BD Pharmingen | 553758 |
| TSG101 | Abcam | ab125011 |
| Albumin | Abcam | Ab137885 |
| Transferrin | Abcam | ab82411 |
| Adiponectin | Abcam | Ab22554 |
| Mouse Complement Factor | R&D systems | MAB5430 |
| D/Adipsin | | |

Chemicals, Peptides, and Recombinant Proteins

| | | |
|---|---|---|
| Dextrose | Hospira, Inc | Cat# |
| | | 0409-6648-02 |
| Insulin | Eli Lilly | NDC 0002-8215-01 |
| Proteinase K | Sigma | Cat# P2308 |
| Poly-L-lysine | Sigma-Aldrich | Cat# P9155 |
| AraC | Sigma | Cat# C6645 |
| DNaseI | Sigma | Cat# DN25 |
| Opti-MEM | GIBCO | Cat# 31985 |
| BODJPY TR ceramide | Thermo Fisher | Cat# D7540 |
| shNAMPT lentivirus particle | Sigma | TRCN0000101276 |

Critical Commercial Assays

| | | |
|---|---|---|
| RNeasy Mini Kit | QIAGEN | Cat# 74104 |
| High-Capacity cDNA Reverse | Thermo Fisher | Cat# 4368814 |
| Transcription Kit | | |
| Total Exosome Isolation | Thermo Fisher | Cat# 4484450 |
| Kit (from plasma) | | |
| Exosome Spin Column | Thermo Fisher | Cat# 4484449 |
| His-3xFlag-NAMPT | N/A | |

Experimental Models: Cell Lines

| | | |
|---|---|---|
| Cell line: HEK293 | ATCC | CRL-3216 |
| Cell line: OP9 | ATCC | CRL-2749 |

Experimental Models: Organisms/Strains

| | | |
|---|---|---|
| Mouse: C57BL/6J | The Jackson | RRID: |
| | Laboratory | IMSR_JAX: 000664 |
| Mouse: Aged C57BL/6J | NIA aging colony | |
| Mouse: ANKI | N/A | |

Software and Algorithms

| | |
|---|---|
| Prism 5 | GraphPad |

### Animal Models

C57BL/6J mice were bred in our laboratory using mice purchased from Jackson Laboratories or obtained from the NIH aged rodent colony. Young (4-6 month-old) and aged (18-26 month-old) mice used in each experiment were age- and source-matched. Cre-inducible STOP-Nampt mice and adiponectin-Cre mice were provided by Joseph Baur at University of Pennsylvania and Evan Rosen at Beth Israel Deaconess Medical Center, respectively. All lines were backcrossed to the C57BL/6J background. For the entire study, heterozygous ANKI mice were generated by crossing heterozygous Adiponectin-Cre mice and homozygous STOP-Nampt mice. Both male and female ANKI mice were used for their characterizations including eNAMPT and tissue NAD+ quantifications, wheel-running analysis, sleep fragmentation counts, ERG analysis, and lifespan. Only male ANKI mice were used for gluco-metabolic and islet morphometric analyses due to their more robust phenotypes. All mice were fed a standard chow diet (LabDiet 5053; LabDiet, St. Louis, MO) ad libitum and housed at 22° C. on a 12/12-hour light/dark cycle in a group of 4-5 unless noted otherwise. Cages and beddings were changed once per week. Mice were monitored periodically for their health status, and there were no viral and parasitic infections during our study.

### Human Subjects

Human plasma samples used for eNAMPT quantification were obtained from male subjects with age ranging from 37 to 80.

### Cell Culture

HEK293 were obtained from ATCC (Manassas, VA) and maintained in DMEM (Sigma Aldrich, St. Louis, MO) supplemented with 10% FBS, 100 U/ml penicillin, and 100 μg/ml streptomycin. OP9 preadipocytes were maintained in α-MEM (Sigma Aldrich, St. Louis, MO) supplemented with 20% FBS and penicillin-streptomycin. All cells were maintained at 37° C. and 5% CO2. OP9 preadipocytes were differentiated into fully differentiated adipocytes by culturing in α-MEM with 0.2% FBS, 175 nM insulin, 900 μM oleate bound to albumin for 48 hrs. Primary hypothalamic neurons were isolated from E14 embryo and cultured in neurobasal media (Sigma Aldrich, St. Louis, MO) supplemented with 10% FBS, 2 mM L-glutamate, and B27. HEK293 was derived from a female fetus. The sex of a mouse from which OP9 preadipocytes were derived is not known. Primary hypothalamic neurons were isolated from both sexes of embryos.

### Lifespan and Hazard Rate Analyses

All animals were kept in our animal facility with unlimited access to standard laboratory diet and water. Mice set aside for the survival study were not used for any other biochemical, physiological, or metabolic analyses. All mice in the aging cohorts were carefully inspected daily. The endpoint of life was determined when each mouse was either found dead or euthanized according to our IACUC guidelines. Necropsy was conducted immediately following the death or euthanasia by the Washington University Mouse Pathology Core. Age-associated mortality rate (qx) was calculated by the number of animals alive at the end of each interval over the number of animals at the beginning of the interval. The hazard rate (hz) was calculated by hz=2 qx/(2−qx), and natural logarithm of hz was plotted against time.

### Physical Activity

Assessment of locomotor activity was performed at the Washington University Animal Behavior Core. Briefly, individual mice were placed into a transparent polystyrene container surrounded by pairs of 4×8 matrix of photocells which quantified total number of ambulation. Another sets of photocells were located 7 cm above the floor to quantify vertical rearing motion. Assessment of wheel-running activity was performed by placing mice in individual cages with a running wheel and housed in circadian cabinetry under 12:12 light-dark cycle. Mice were habituated for 2 weeks before wheel-running activity measurements.

### Sleep Analysis

Mice were anesthetized with isofluorane and surgically implanted with screw electrodes in the skull for electroencephalography (EEG) and stainless wire electrodes in the nuchal muscle for electromyography (EMG). Mice were recovered from surgery for three days and subsequently habituated in the recording cage for two weeks. EEG/EMG recording was performed continuously for 2 consecutive days. 10-second epochs of EEG/EMG signals were visually scored as wake [low amplitude delta (1-4 Hz) and theta (4-8 Hz) frequency with high EMG activity], NREM sleep [high amplitude delta in the absence of EMG activity], and REM sleep [low amplitude rhythmic theta activity in the absence of EMG activity]. Scorer was blinded for genotypes during quantification.

### Metabolic Assessments

For glucose tolerance tests, mice were intraperitoneally injected with one dose of dextrose (1 g/kg body weight) after overnight fasting in aspen bedding. Blood was collected from the tail vein at each time point for the measurement of blood glucose and insulin levels. For insulin tolerance tests, mice were intraperitoneally injected with insulin (0.70 units/kg body weight), and blood was collected from tail vein for the measurement of blood glucose. Quantification of plasma insulin levels was performed using the Singulex assay at the Core Laboratory for Clinical Studies at Washington University. EchoMRI was performed at the Diabetes Models Phenotyping Core of the Diabetes Research Center at Washington University.

### Electroretinography

Mice anesthetized by a mixture of ketamine and xylazine were subjected to ERG using UTAS-E3000 Visual Electrodiagnostic System. Quantitation of ERG waveforms were performed using an existing Microsoft Excel macro that defines a-wave amplitude as the difference between the average baseline and the most negative point of the average trace and also defines b-wave amplitude as the difference between the most negative point to the highest positive point of the wave peak.

### Small Cohort Prospective Lifespan Analysis

Female C57BL/6J mice at 26-28 months of age were obtained from the NIA aging colony. Plasma was collected from the tail vein blood to quantify eNAMPT levels. Subsequently, the mice were housed in groups of 4 mice per cage and untouched except for daily inspection. The number of days from the blood collection to the death was calculated as a remaining lifespan.

Western Blot Analysis of eNAMPT

Plasma was collected from the tail vein by capillary or cardiac puncture with syringe pre-treated with heparin sulfate under ketamine-xylazine anesthetization. Blood was span down at 3000×g. 2 μl of freshly collected plasma was incubated with 200 μl of 1× sample buffer at 95° C. for 10 min before being stored at −30° C. until its use. Right before the analysis, 5 μl of each sample was added to 45 μl of 1× sample buffer and further incubated at 95° C. for 30 min. This 30-min boiling was necessary to make eNAMPT bands discrete and quantifiable. Plasma eNAMPT was detected as doublets when the run time of SDS-PAGE was long enough. 10 μl of the final mixture was separated on 4-15% SDS-PAGE and analyzed by Western blotting with anti-NAMPT polyclonal antibody (Bethyl) for mice and anti-NAMPT monoclonal antibody (Adipogen) for humans. NAMPT antibodies were used at 1:1000 dilutions. All other antibodies were used at 1:100 dilutions.

### Gene Expression Analysis

RNA was extracted by RNeasy Mini Kit (QIAGEN) and converted to cDNA by High-Capacity cDNA Reverse Transcription Kit (Thermo). Quantitative real-time RT-PCR was conducted with the StepOnePlus system (Applied Biosystems), and relative expression levels were calculated for each gene by normalizing to Gapdh levels and then to the average of the control mice.

### EV Purification and Characterization

EVs used in this study were isolated using ultracentrifugation or the Total Exosome Isolation Kit From Plasma (ThermoFisher Scientific) according to the manufacturer's instruction. Mouse plasma was isolated by centrifuging blood at 1,000×g for 10 min. EVs were also collected in vitro by conditioning serum free α-MEM with fully differentiated OP9 adipocytes for 48 hrs. Isolated plasma and OP9 conditioned media were centrifuged at 1000×g for 10 min. Supernatant was centrifuged again for 2000× g for 20 min. The resulting supernatant was further centrifuged at 10,000×g for 30 min prior to EV isolation.

For EV isolation from plasma by ultracentrifugation, plasma was diluted 1:1 in PBS and centrifuged at 100,000×g for 2 hrs. The resulting supernatant was collected for western blot analysis and remaining pellet was resuspended into the volume of PBS equal to the starting plasma volume and centrifuged again at 1000,000×g for 2 hrs. For EV isolation from OP9 conditioned media by ultracentrifugation, media was centrifuged at 100,000×g for 2 hrs. Again, resulting supernatant was collected for western blot analysis, and the remaining pellet was resuspended into the volume of PBS equal to the starting volume of media. Resuspended EVs were centrifuged again at 100,000×g for 2 hrs. The final resulting pellet of EVs from both plasma and OP9 conditioned media was resuspended in 50 μl of PBS.

For EV isolation by the Total Exosome Isolation (TEI) kit, EVs were resuspended in the same volume of PBS as the volume of plasma used to isolate EVs unless noted otherwise. The resulting supernatant subsequent to EV isolation was collected as the soluble protein fraction. The quality of isolated EVs was confirmed by measuring the levels of EV marker proteins [Alix (Santa Cruz Biotechnology), TSG101 (Santa Cruz Biotechnology), CD63 (Santa Cruz Biotechnology), CD81 (Santa Cruz Biotechnology) and CD9 (BD Bioscience)], and non-EV proteins [transferrin (abcam), albumin (abcam), adiponectin (abcam), and adipsin (R&D)]. 40 μg of protein from total plasma, isolated EV fraction, and supernatant/soluble protein fraction were loaded to a SDS-PAGE gel and evaluated by Western blotting.

### Sucrose Gradient Fractionation Analysis of EVs

EVs were prepared by either ultracentrifugation at 100,000×g for 2 hrs or by the TEI kit. The isolated EVs were diluted with 90% sucrose solution to a final concentration of 82%. The EVs were then layered at the bottom, and subsequently, sucrose solutions ranging from 82%-10% were layered above. Samples were centrifuged at 100,000×g for 20 hrs, and 6 fractions were collected. Each fraction was diluted 1:100 in PBS and centrifuged at 100,000×g for 2 hrs to pellet EVs. A pellet from each fraction was then resuspended in an equal volume of PBS and subjected to the analysis by Western blotting.

### Proteinase K Digestion Assay

Proteinase K was added to 50 μl of plasma at the final concentration of 1 μg/μl and incubated at 37° C. for 10 min. Subsequently, 25 μl of PBS and 15 μl of the Exosome Precipitation Reagent (ThermoFisher Scientific) were added, and the mixture was incubated on ice for 30 min. The mixture was centrifuged at 1000×g, and the precipitated EVs were analyzed by Western blotting.

### Proteomic Analysis of Plasma EVs

Plasma was isolated from EDTA-supplemented blood isolated from 6 and 24 month-old wild-type B6 female mice and 24 month-old control and ANKI female mice. EVs were isolated from 400 μl of plasma by ultracentrifugation and reconstituted in water. Proteins were extracted and analyzed by Progenesis LC-MS (NonLinear Dynamics). Protein identification was done with Mascot Server v2.4 (Matric Science). A list of identified proteins was generated with peptide threshold with 95% minimum, protein threshold with 95% minimum and 2 peptides minimum, and protein false discovery rate at 0.5%. Out of 248 proteins identified with above threshold, 181 proteins were identified in the past proteomic study of EVs/exosomes, based on EVpedi.org.

### Isolation of Primary Hypothalamic Neurons

Hypothalamicrom E16-E18 embryos were dissected and placed on ice in the Hibernate E medium. Hypothalamicere digested in 0.25% trypsin-EDTA (Sigma) supplemented with DNase I (Sigma) at 37° C. for 15 min. After an equal volume of DMEM supplemented with 10% FBS was added, cells were gently dissociated by pipetting until no clumps remained. Cells were collected by centrifuging at 450× g for 5 min at room temperature. Cells were washed and resuspended in the Neurobasal media containing 10% FBS, 2% B27, 2M L-glutamine, and antibiotics. Cells were plated onto wells directly or onto coverslips pre-coated with poly-1-lysine (Sigma). Two days after the isolation, cells were treated with 10 μM Ara-C(Sigma) for at least 4 days or until non-neural cells were eliminated.

### EV Internalization Assay

Isolated EVs were resuspended in PBS. BODIPY TR ceramide in DMSO was added to EVs or PBS at the final concentration of 100 μM and incubated at 37° C. for 1 hr. Unincorporated dye from the labeled EVs was removed by Exosome Spin Column (ThermoFisher Scientific) following manufacturer's instructions. Purified EVs or PBS solution was added directly to primary hypothalamic neurons growing on coverslips and incubated for 30 min. Following incubation, cells were washed in PBS and fixed in 4% paraformaldehyde.

Generation of Recombinant NAMPT-Containing EVs and their Internalization Assay

Isolated EVs were resuspended in 1 μg/μl FLAG-tagged recombinant NAMPT protein (recNAMPT) and incubated overnight at 37° C. recNAMPT-containing EVs were isolated from the mixture by adding 0.2 volume of Exosome Precipitation Reagent (ThermoFisher Scientific). recNAMPT-containing EVs were reconstituted into the same volume of PBS as that of the starting plasma.

Wheel-Running Assay after EV Injection

For EV injection experiments, 20 month-old male and female mice were habituated by 6 days of mock injection. For pre-treatment measurements of wheel-running activity, mice were intraperitoneally injected with 100 μl of PBS for 4 days. Subsequently, the same mice were injected with 100 μl of resuspended EVs purified from 200 μl plasma collected from 4-6 month-old mice and resuspended in PBS. Every injection was performed approximately at 5:30 μm.

### Lifespan Study of EV-Injected Mice

25 month-old female C57BL/6J mice were obtained from the National Institute on Aging (NIA). Mice were sorted by their weights, and pairs of mice with similar body weight were allocated to each group. Four mice were housed per cage. EVs were isolated from plasma of 4-12 month-old wild-type mice by the TEI kit. In this lifespan study, the use of the TEI kit was necessary to achieve the highest yields of EVs from the limited numbers of available mice. EVs isolated from 500 μl of plasma were resuspended in 100 μl of PBS and administered to mice once a week by intraperitoneal injection, starting at 26 months of age.

### Data Analysis

Results are presented as mean±SEM. All statistical tests were performed using GraphPad Prism 5. Significance between two groups was assessed by Student's t test. Normality of the data was assessed graphically. The comparisons between multiple groups were carried out using one-way ANOVA with Tukey posthoc test. Analysis of plasma eNAMPT levels over 24 hrs between 6 and 18 month-old mice was performed using two-way repeated measures ANOVA. Linear regression analysis was used to analyze plasma eNAMPT levels of mice and humans across different age groups. Comparison of locomotor and wheel-running activities was performed by Wilcoxon matched-pairs singled-ranked test. ERG signal was analyzed by two-way repeated measures ANOVA with Bonferroni posthoc test. Gehan-Breslow-Wilcoxon test was used for the statistical analysis of lifespan. Fisher's exact test was used to compare the proportion of the cause of death. Statistical comparison of wheel-running activities in pre- and post-treatments with EV injection was performed by a paired t test. Sample sizes and other statistical parameters are indicated in the figures and texts. *p<0.05, **p<0.01, ***p<0.001. Significance was concluded at p<0.05.

### Example 1: Plasma eNAMPT Levels Decline with Age in Both Mice and Humans

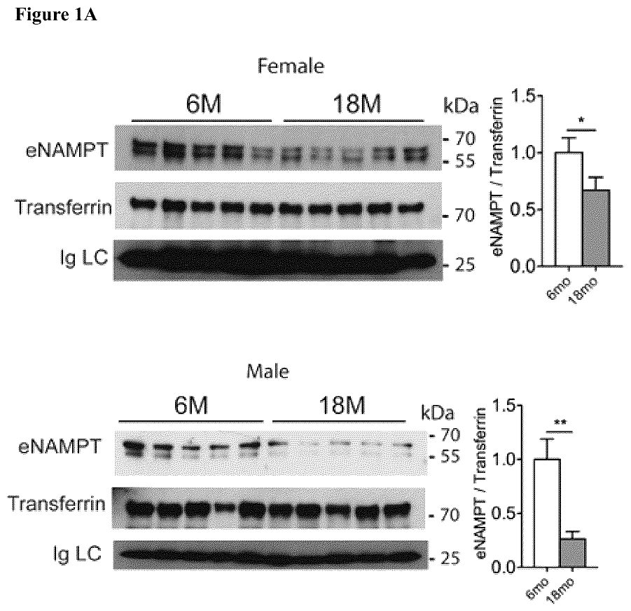

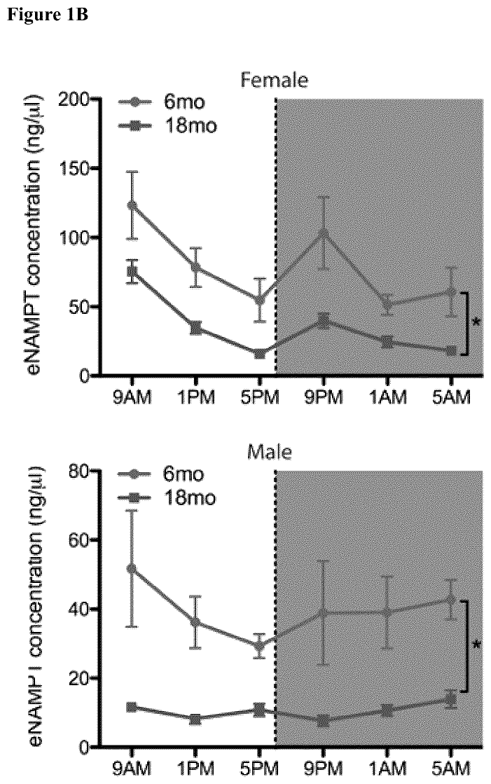

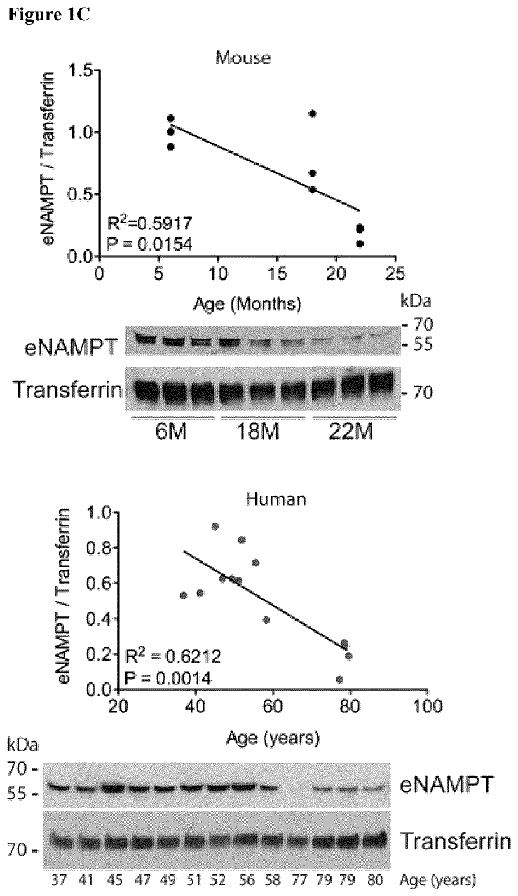

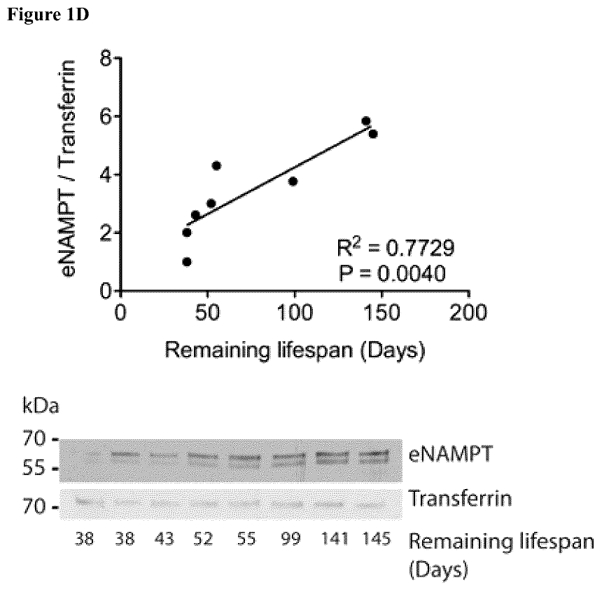

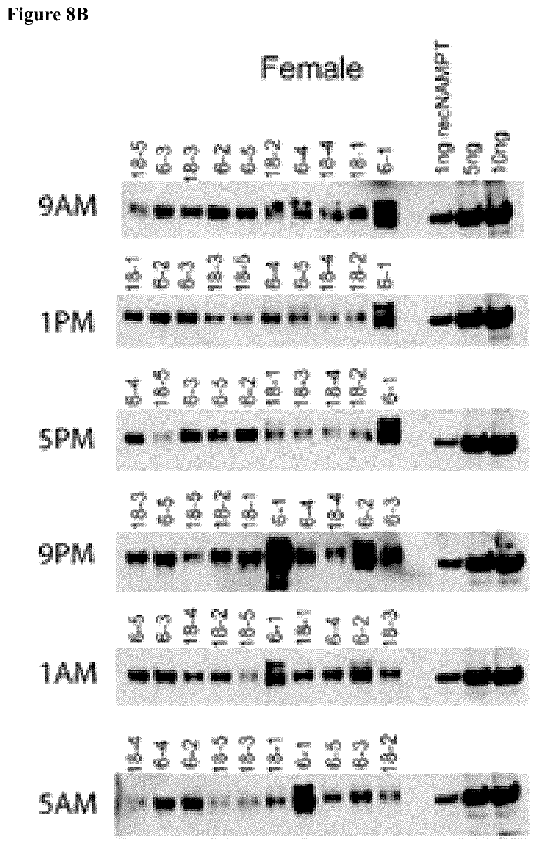

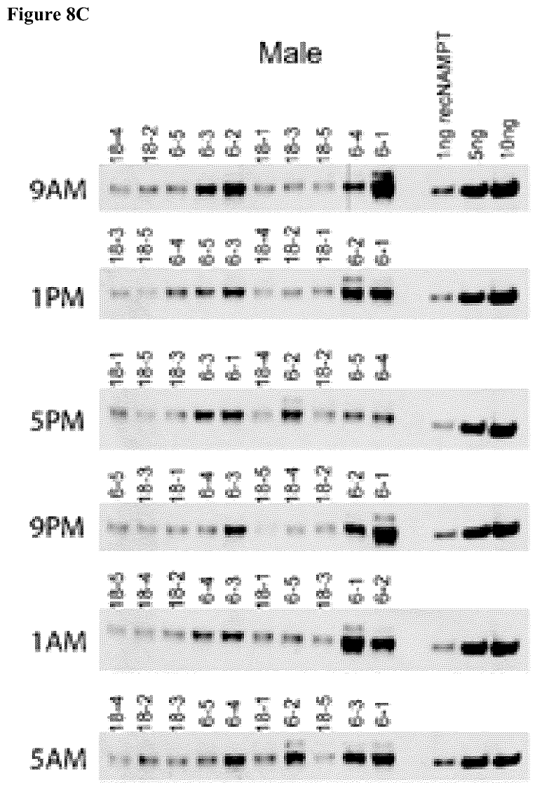

Our previous study has demonstrated that adipose NAMPT expression decreases with age (Yoshino et al., 2011). Consistently, we found that the protein expression levels of iNAMPT in isolated adipocytes decreased from 6 months to 18 months of age (FIG. 8A). Given that adipose tissue is a major source of circulating eNAMPT (Yoon et al., 2015), we examined whether circulating eNAMPT levels changed during aging in mice. Plasma eNAMPT levels significantly declined from 6 months to 18 months of age by 33% and 74% in both female and male mice, respectively (FIG. 1A). In young (6 month-old) mice, calculated plasma eNAMPT concentrations were higher in females (55-123 ng/μl) compared to those in males (29-52 ng/μl). However, in aged (18 month-old) mice, both males and females showed significant decline in circulating eNAMPT levels throughout a day (FIGS. 1B and 8B).

The age-associated reduction in plasma eNAMPT raised a possibility that plasma eNAMPT levels could be a valuable surrogate biomarker for aging. Thus, we measured plasma eNAMPT levels across several different age groups in mice and humans. We found that plasma eNAMPT levels linearly declined with age in both mice and humans (FIG. 1C), suggesting that the process underlying eNAMPT secretion and its potential significance during aging might be conserved in both species. To further evaluate the potential association between plasma eNAMPT levels and aging, we asked whether the reduced levels of plasma eNAMPT could predict higher mortality risks and remaining lifespans of mice in a small prospective study. Intriguingly, the number of days for which an individual mouse lived since the day of eNAMPT measurement was highly correlated with their plasma eNAMPT levels (FIG. 1D). The higher the level of plasma eNAMPT went, the longer the remaining lifespan became. These results led us to the interesting hypothesis that circulating eNAMPT might play a critical role in regulating not only the process of aging and but also lifespan in mammals.

### Example 2: Adipose-Tissue Specific Overexpression of Nampt Maintains Plasma eNAMPT Levels and NAD+ Biosynthesis in Multiple Tissues During Aging

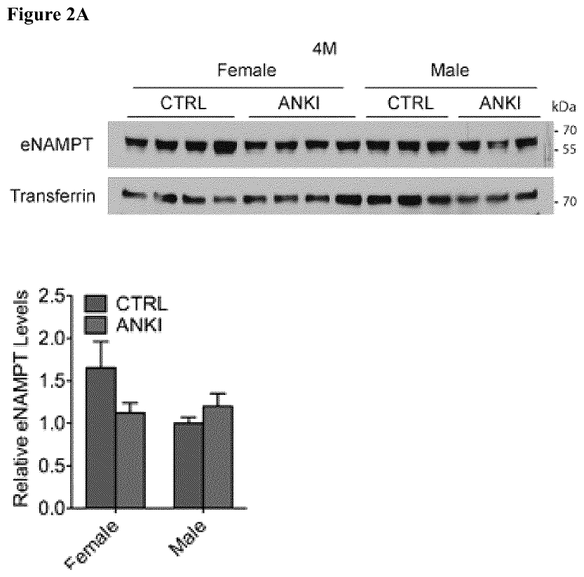

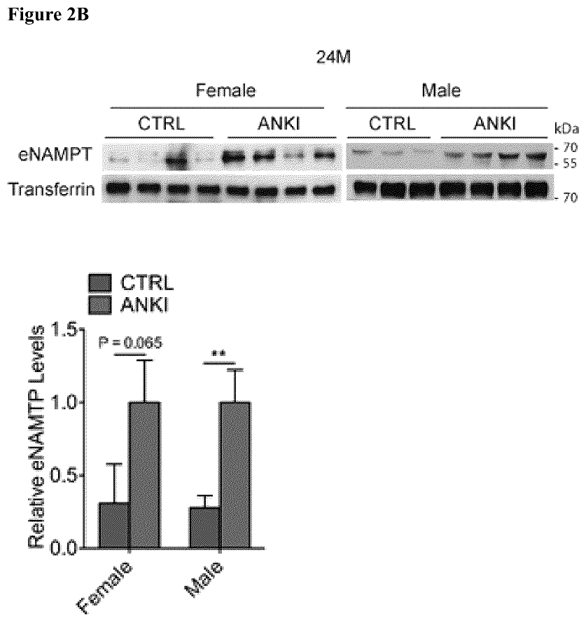

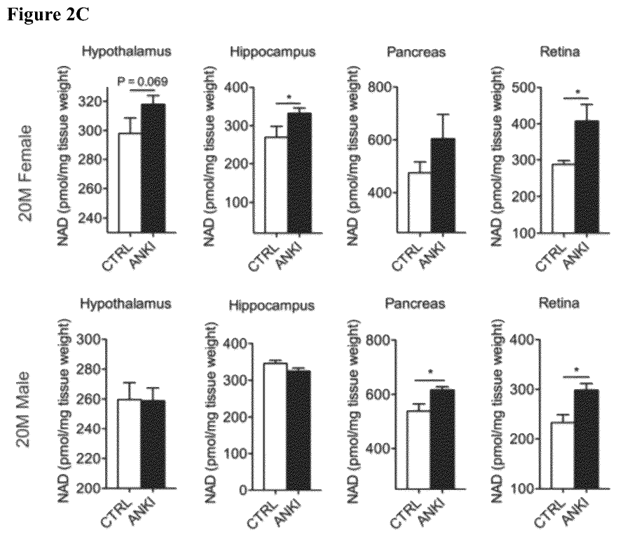

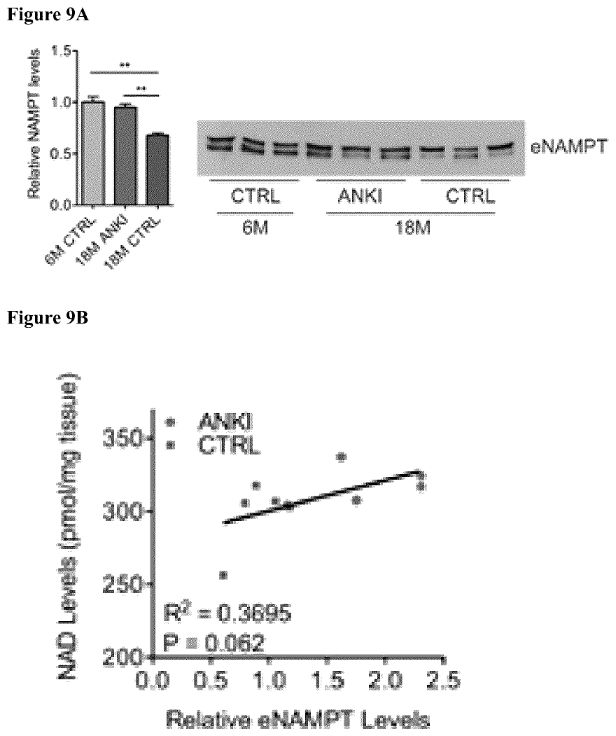

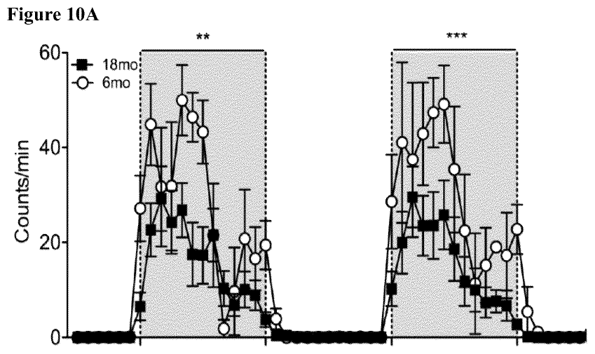

To investigate the role of eNAMPT in aging and longevity control, we examined aging cohorts of adipose tissue-specific Nampt knock-in (ANKI) mice (Yoon et al., 2015). At 4 months of age, plasma eNAMPT levels did not differ between ANKI and control mice under an ad libitum-fed condition (FIG. 2A). Young ANKI mice showed significantly higher levels of plasma eNAMPT only in response to fasting (Yoon et al., 2015). When they reached 24 months of age, plasma eNAMPT levels were maintained at 3.3- and 3.6-fold higher levels in ANKI female and male mice, respectively, compared to those in the age-matched control mice (FIG. 2B). Plasma eNAMPT levels in 18 month-old ANKI mice were comparable to those in 6 month-old control mice (FIG. 9A). Adipose tissue Nampt overexpression was maintained ~1.5-fold higher in aged ANKI mice, compared to that in control mice, confirming that Nampt overexpression was in the physiological range and suggesting that the ANKI model is physiologically valid (data not shown).

In ad libitum-fed 20 month-old ANKI mice, increased NAD+ levels were observed in the hypothalamus, hippocampus, pancreas, and retina in females, whereas only pancreas and retina showed increased NAD+ levels in males (FIGS. 2C and 9B). It should be noted that those tissues that showed increased NAD+ levels in aged ANKI mice are the ones that have relatively very low levels of iNAMPT (Revollo et al., 2007; Stein et al., 2014; Yoon et al., 2015). These results suggest that adipose tissue-specific overexpression of Nampt mitigates age-dependent decline in circulating eNAMPT levels and tissue NAD+ levels in multiple tissues including the hypothalamus, hippocampus, pancreas, and retina.

### Example 3: Aged ANKI Mice Display Significant Enhancement in Physical Activity and Sleep Quality

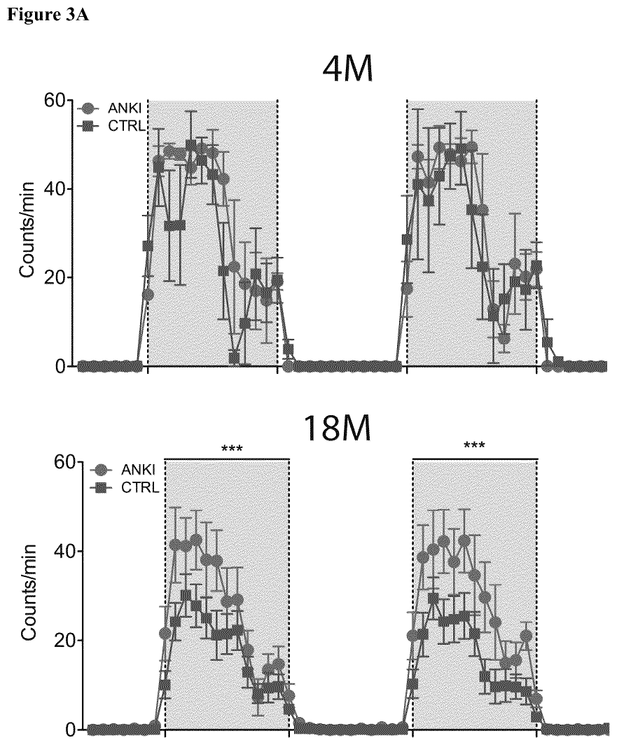

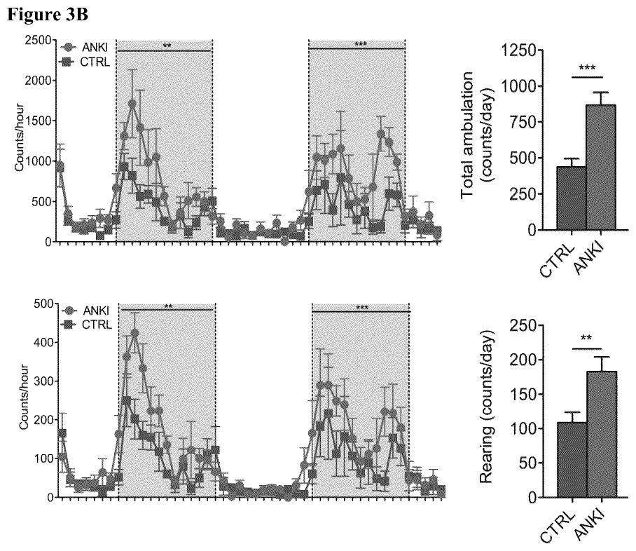

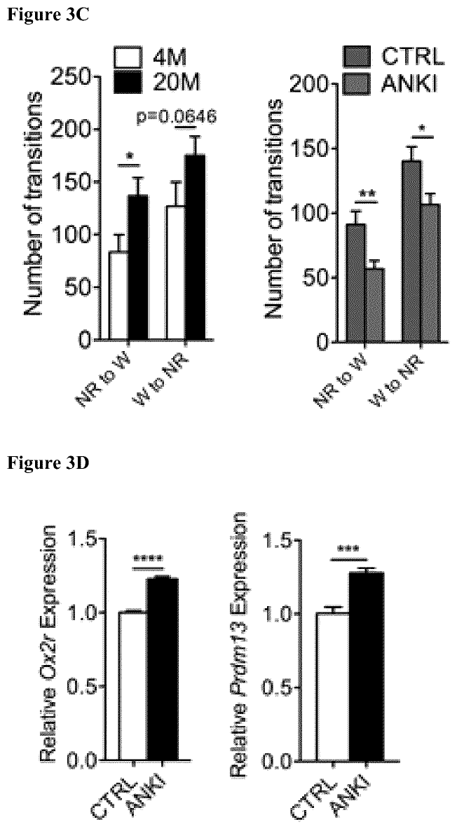

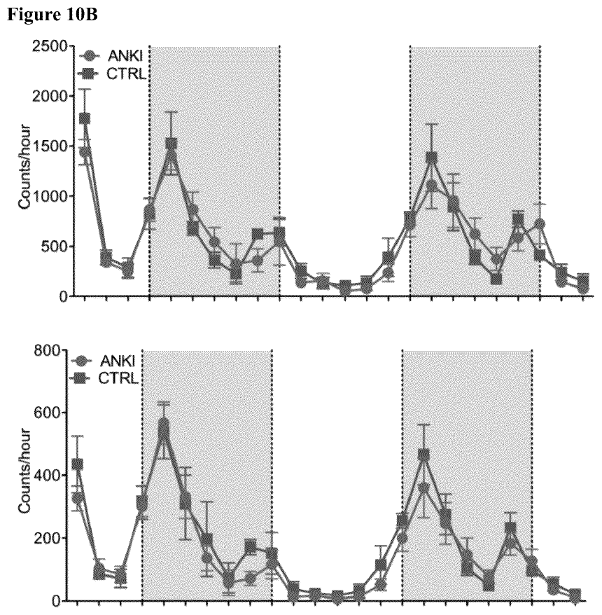

Given that hypothalamic NAD+ levels increased in aged ANKI female mice and also that hypothalamic SIRT1 activity is critical to regulate physical activity and sleep quality during aging (Satoh et al., 2013; Satoh et al., 2015), we examined these age-associated physiological traits in aged ANKI mice. Consistent with the reduction in circulating eNAMPT levels with age, wheel-running activity during the dark time was significantly reduced in 18 month-old wild-type mice, compared to that in 6 month-old wild-type mice (FIG. 10A). ANKI female mice at 4 months of age exhibited equivalent levels of wheel-running activity during the dark time to those of age-matched control mice, whereas ANKI female mice at 18 months of age showed significantly enhanced wheel-running activity compared to that in the age-matched control mice, similar to the activity levels in 6 month-old wild-type mice (FIGS. 3A and 10A). Additionally, we evaluated locomotor activity in the open field in these mice. Consistent with wheel-running activity during the dark time, aged ANKI female mice showed significantly higher total ambulatory and rearing activities, compared to the age-matched control mice (FIG. 3B). However, aged ANKI male mice failed to show any significant differences in total ambulatory and rearing activities, compared to the age-matched control mice (FIG. 10B), consistent with the lack of NAD+ increase in the hypothalamus (FIG. 2C).

In humans, it has been well documented that the number of sleep-wake transitions increases over age, a phenomenon called sleep fragmentation (Mander et al., 2017). Consistent with such changes in the older humans, 20 month-old wild-type mice also showed increased numbers of transitions between non-REM (NREM) sleep and wake cycles, compared to those in 4 month-old wild-type mice (FIG. 3C, left panel), indicating that sleep fragmentation significantly increases with age in mice. There were no differences in the numbers of transitions between REM sleep and wake or NREM sleep cycles (data not shown). Interestingly, compared to the age-matched control mice, aged ANKI mice showed a significant reduction in the numbers of transitions between NREM sleep and wake cycles, which maintained levels similar to those seen in 4 month-old wild-type mice (FIG. 3C, right panel), implying that aged ANKI mice maintain a better quality of sleep.

These age-associated activity and sleep traits are regulated by hypothalamic SIRT1 through the regulation of its downstream target genes, Orexin type-2 receptor (Ox2r) and PR domain 13 (Prdm13) (Satoh et al., 2013; Satoh et al., 2015). Ox2r expression is important for the control of wheel-running activity during the dark time (Satoh et al., 2013), whereas Prdm13 expression is critical for the maintenance of sleep quality (Satoh et al., 2015). Thus, we examined mRNA expression levels of Ox2r and Prdm13 in the hypothalamicf age-matched control and ANKI female mice. Consistent with the observed enhancement of physical activity and sleep quality, hypothalamic Ox2r and Prdm 13 expression levels were significantly increased in aged ANKI female mice, compared to those in age-matched control mice (FIG. 3D). These results indicate that an age-associated decline in circulating eNAMPT levels contributes to the reduction in hypothalamic NAD+ levels and SIRT1 activity, resulting in the age-associated decline in physical activity and sleep quality, and that these functional reductions can be ameliorated by increasing circulating eNAMPT.

### Example 4: Aged ANKI Mice Show Significant Improvement in Glucose-Stimulated Insulin Secretion, Photoreceptor Function, and Cognitive Function

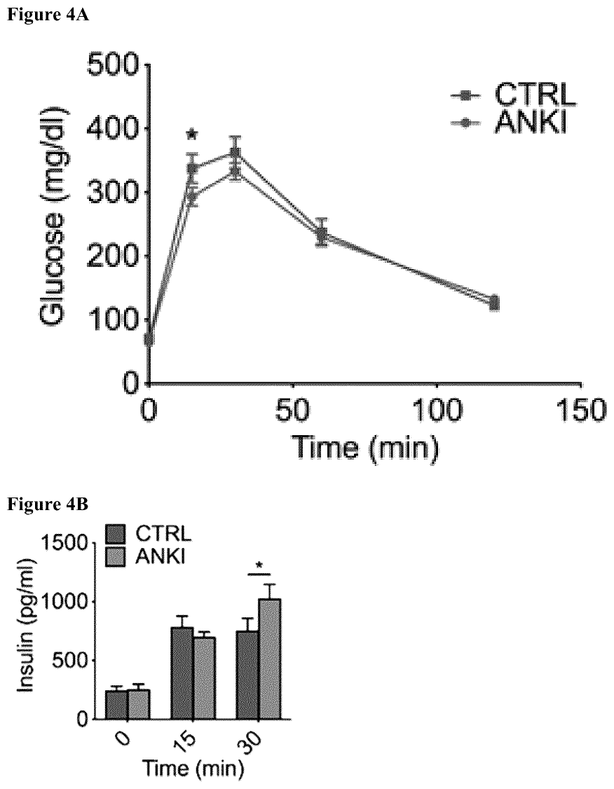

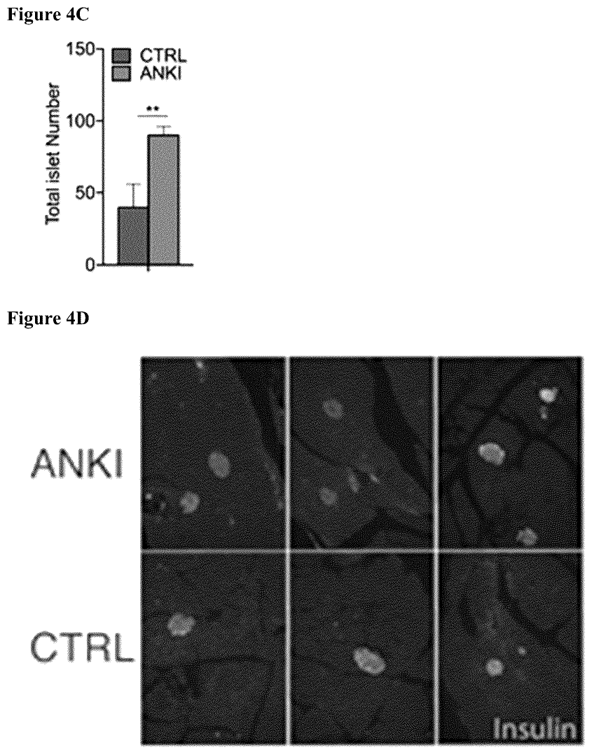

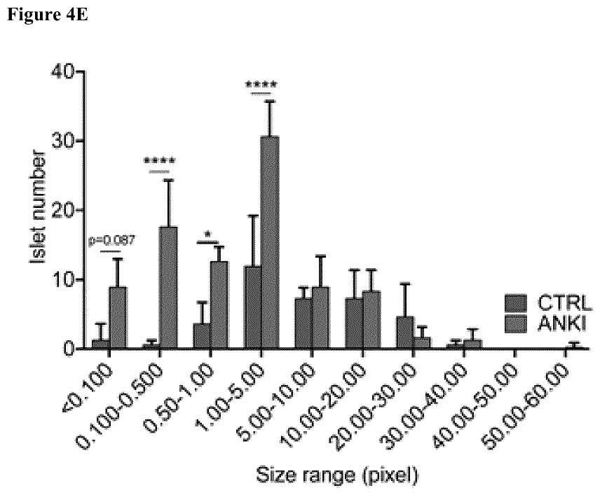

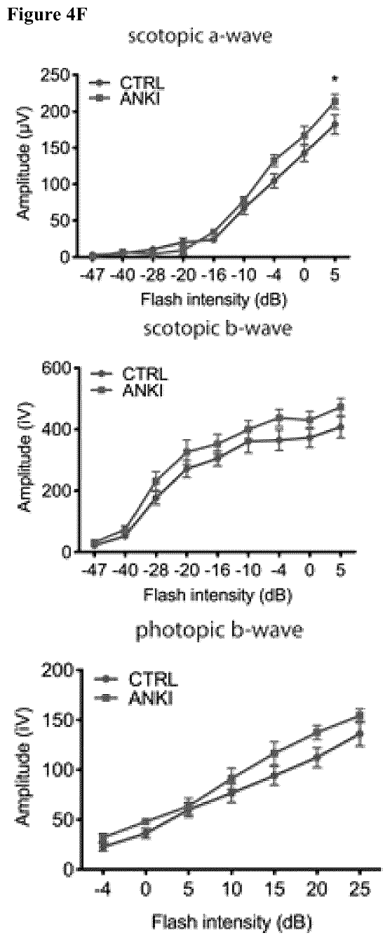

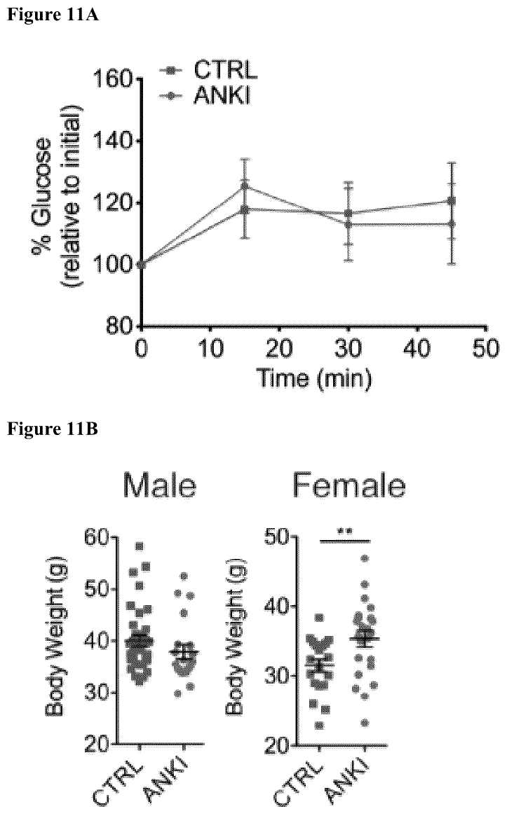

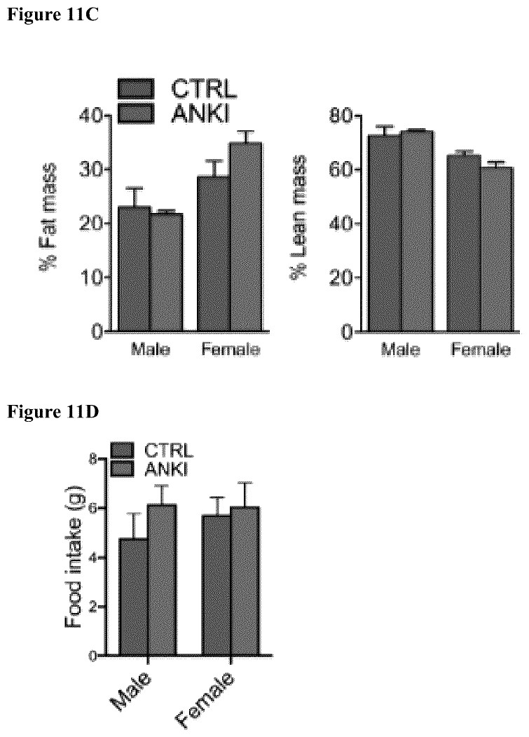

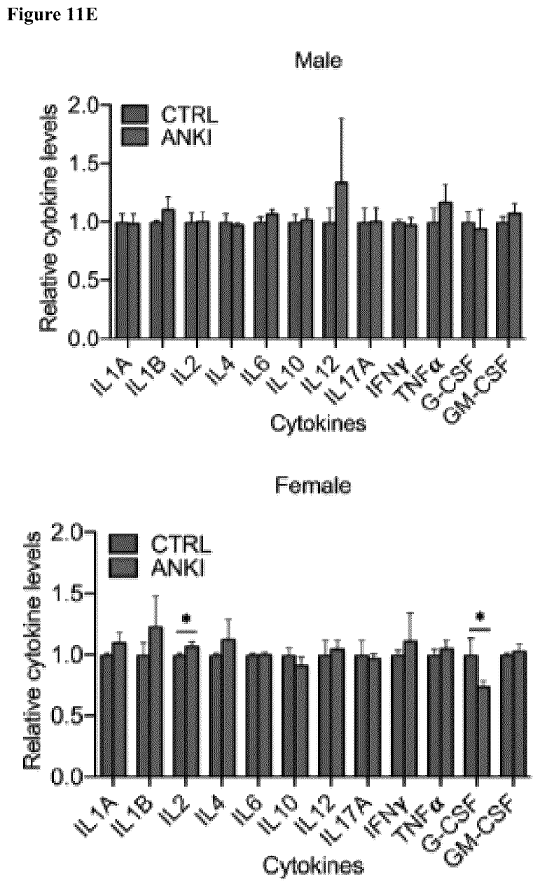

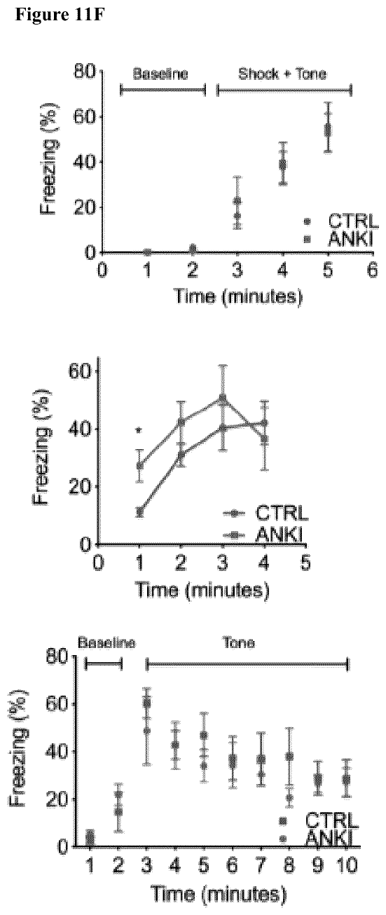

Because pancreatic, retinal, and hippocampal NAD+ levels were increased in aged ANKI mice (FIG. 2C), we suspected that they might also exhibit improvements in glucose metabolism, retinal function, and cognitive function. We first performed intraperitoneal glucose tolerance tests (IPGTTs) in aged ANKI and control mice. We observed a moderate but significant improvement in glucose tolerance in aged ANKI male mice (FIG. 4A). During IPGTTs, we also detected a significant increase in glucose-stimulated insulin secretion at the 30-min time point (FIG. 4B). The results from insulin tolerance tests did not differ between aged ANKI and control mice, both showing severe insulin resistance (FIG. 11A), suggesting that improved glucose tolerance in aged ANKI mice is mainly due to increased insulin secretion. Interestingly, aged ANKI mice possessed a higher total number of islets, compared to age-matched controls (FIGS. 4C and D). Additionally, those islets observed in aged ANKI males were kept smaller than those in age-matched controls (FIG. 4E). These results are consistent with the role of NAD+ biosynthesis and SIRT1 in promoting glucose-stimulated insulin secretion in pancreatic β cells and protecting them from stresses (Kitamura et al., 2005; Moynihan et al., 2005; Ramsey et al., 2008; Revollo et al., 2007). Aged ANKI female mice also maintained a higher total number of islets but did not show any improvement in glucose tolerance (data not shown), consistent with a much larger variability in pancreatic NAD+ levels (FIG. 2C). Aged ANKI female mice showed moderate increases in body weight and fat mass, whereas aged ANKI male mice showed no difference (FIGS. 11B and 11C). Furthermore, their food intake did not show any significant differences, compared to their age-matched controls (FIG. 11D). We also examined circulating levels of proinflammatory cytokines in aged ANKI mice. There were no significant changes in circulating proinflammatory cytokine levels in both aged ANKI males and females, except for slight increases in IL-2 and moderate decreases in G-CSF in aged ANKI females (FIG. 11E). Thus, the observed ANKI phenotypes in glucose metabolism are unrelated to body weight, adiposity, or proinflammatory cytokine levels.

We next conducted electroretinography to examine retinal function under rod- and cone-dominated testing conditions. We found that, compared to age-matched control mice, aged ANKI mice showed significantly higher scotopic a-wave amplitudes at 5 db and a trend toward higher scotopic a-wave amplitudes at −4 and 0 db. (FIG. 4F, left panel), indicating improved rod photoreceptor function. Similarly, improvement in rod photoreceptor function also led to trends towards enhanced scotopic b-waves (FIG. 4F, middle panel). We also observed trends toward enhanced photopic b-wave amplitudes in aged ANKI mice, which suggest enhanced cone function (FIG. 4F, right panel). Of interest, these changes are remarkably similar to those observed in the long-term NMN administration (Mills et al., 2016).

We also performed contextual fear conditioning tests on aged ANKI and age-matched control mice to assess their nonspatial hippocampus-dependent learning and memory capabilities. Both mice showed equivalent responses during the baseline and training trials on day 1 (FIG. 11F, first graph). However, during the first minute of the contextual fear conditioning test on day 2, aged ANKI mice exhibited significantly higher levels of freezing, compared to age-matched controls (FIG. 11F, second graph). A similar trend was also observed at 2 and 3 min time points during this trial. Throughout the baseline and the auditory cue tests on day 3, aged ANKI and age-matched control mice did not show any significant differences (FIG. 11F, third graph). Consistent with their hippocampal NAD+ increases, these results suggest that aged ANKI mice maintain a better hippocampus-dependent cognitive function, compared to their age-matched controls. Thus, taken together, these findings provide further support to the physiological significance of eNAMPT in the maintenance of tissue functions, such as the hypothalamus, hippocampus, pancreas, and retina, during aging by enhancing systemic NAD+ biosynthesis.

### Example 5: ANKI Female Mice Exhibit Significant Extension of Median Lifespan and Delay in Aging

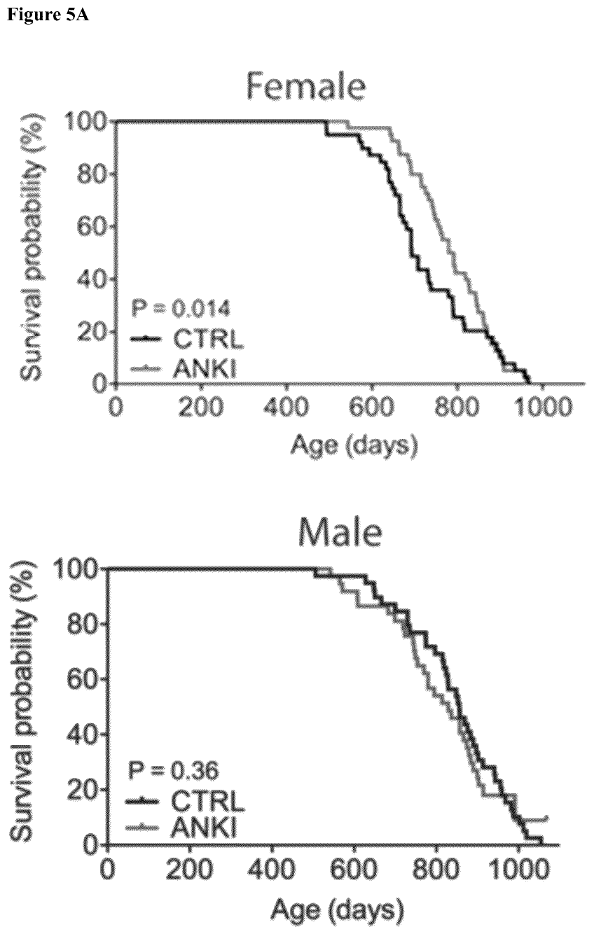

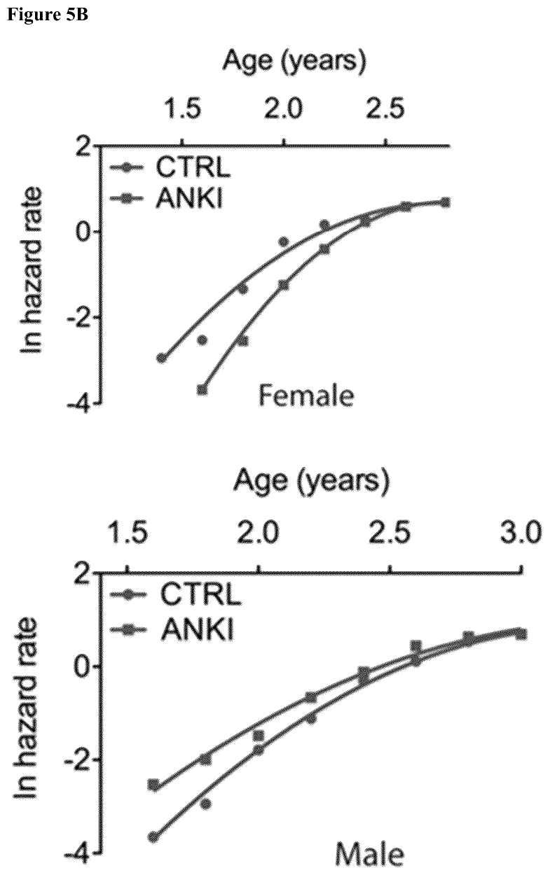

Because maintaining higher eNAMPT levels significantly mitigates age-associated functional decline in aged ANKI mice, we set up cohorts of ANKI and control mice to examine their lifespan. When fed regular chow ad libitum, ANKI female mice showed statistically significant extension (13.4%) of median lifespan (control 693 days versus ANKI 786 days, Gehan-Breslow-Wilcoxon test, □2=6.043, df=1, p=0.014) (FIG. 5A Table 2).

TABLE 2
Lifespan parameters of control and ANKI mice. Mean and maximal lifespans of the oldest 10% and
20% of each cohort are shown as mean values ± SEM. The differences in survival curves and
mean lifespans were assessed by Gehan-Breslow-Wilcoxon test and Student's t test, respectively.

| | | | | | P value by | Min-Max | | |
|---|---|---|---|---|---|---|---|---|
| | | | Mean | Median | Wilcoxon | (in 20% | Oldest | |
| | | n | (days) | (days) | Test | oldest) | 10% | 20% |
| Female | Control | 39 | 728 ± 20 | 693 | 0.014 | 989-967 | 942 ± 13 | 914 ± 13 |
| | ANKI | 40 | 786 ± 15** | 786 | | 871-962 | 933 ± 15 | 909 ± 15 |
| Male | Control | 39 | 844 ± 20 | 856 | 0.56 | 959-1055 | 1022 ± 12 | 998 ± 11 |
| | ANKI | 39 | 826 ± 23 | 856 | | 914-1085 | 1068 ± 13* | 1024 ± 21 |

Their maximal lifespan did not differ from that of control mice (Table 2). Interestingly, ANKI female mice exhibited significant delays in age-associated mortality up to ~2 years of age (FIG. 5B). However, towards the end of their lifespan, the difference in age-associated mortality was no longer present, which could explain the lack of maximal lifespan extension. Although neoplasms are a major cause of death, the incidence and the type of neoplasms did not differ between ANKI and control mice (Table 3).

TABLE 3
Identified causes of death in aged control and ANKI
mice (n = 37). Sarcoma subtypes includes histio-, hemangio-,
lipo-, and fibrosarcoma, and carcinoma subtypes includes
hepatocellular, bronchiolo-alveolar, and cholangiocarcinoma.

| | CTRL | % | ANKI | % |
|---|---|---|---|---|
| Neoplasm | 29 | 67.4 | 26 | 60.5 |
| Sarcoma | 22 | 51.2 | 18 | 41.9 |
| Lymphoma | 3 | 7.0 | 4 | 9.3 |
| Carcinoma | 3 | 7.0 | 2 | 4.7 |
| Leukemia | 1 | 2.3 | 2 | 4.7 |
| Other | 6 | 14.0 | 6 | 14.0 |
| Non-neoplasm* | 8 | 18.6 | 11 | 25.6 |

*Septicemia, urinary tract obstruction, thromboembolism, cardiomyopathy, fecal impaction

In contrast to females, ANKI male mice exhibited no lifespan extension (FIG. 5A and Table 2) and no difference in age-associated mortality rate throughout the majority of their lifespan (FIG. 5B), although the oldest 10% ANKI males showed a significantly longer maximal lifespan compared to control mice. These results demonstrate that maintaining youthful levels of circulating eNAMPT is critical to delay aging and extend healthspan in mice, although there is a significant sex difference.

### Example 6: Plasma eNAMPT is Localized Exclusively to Extracellular Vesicles

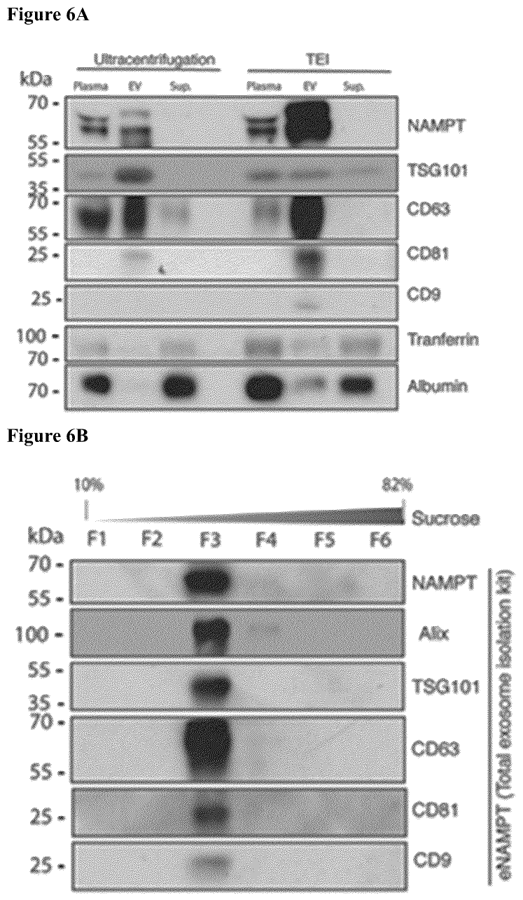

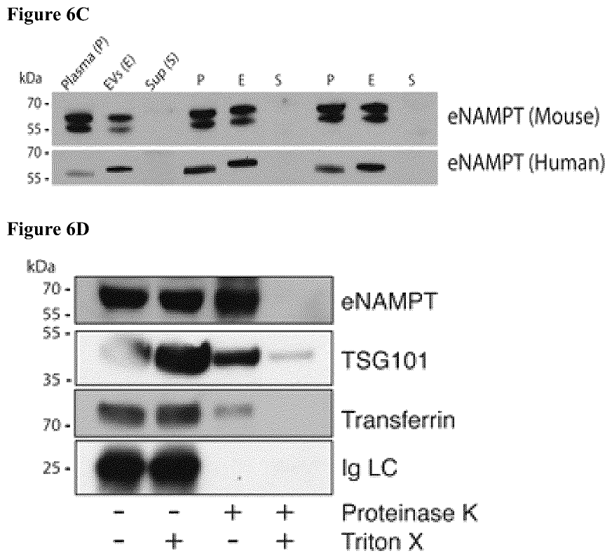

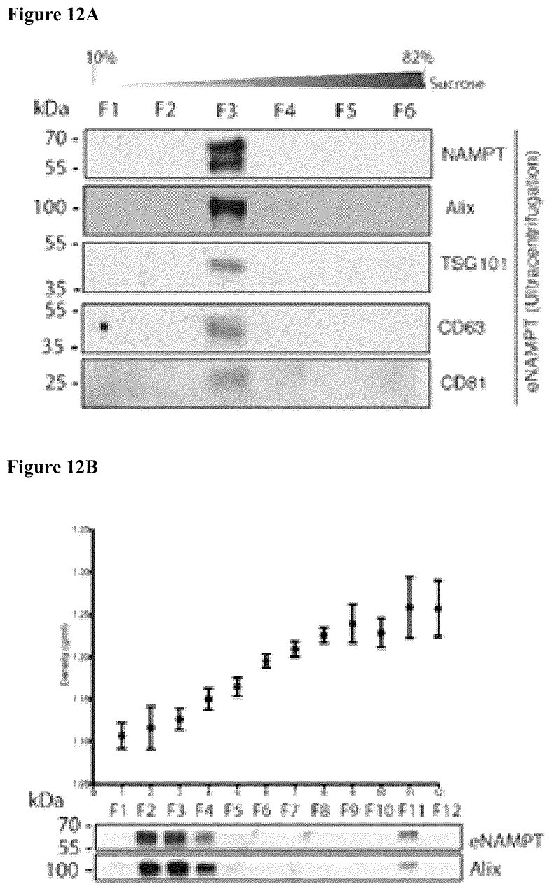

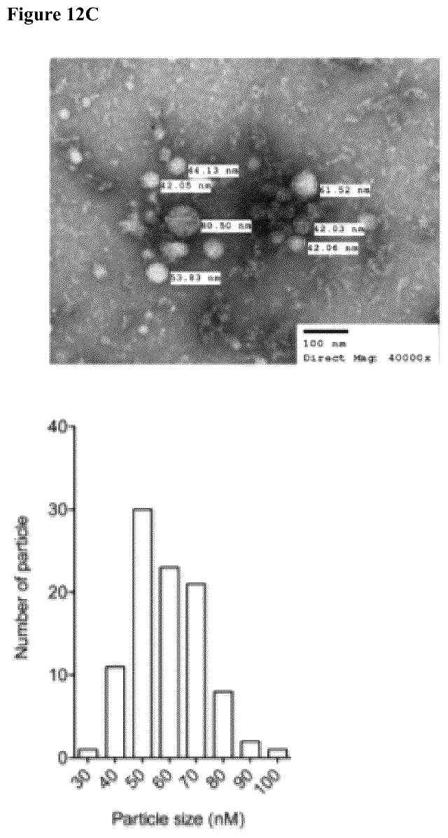

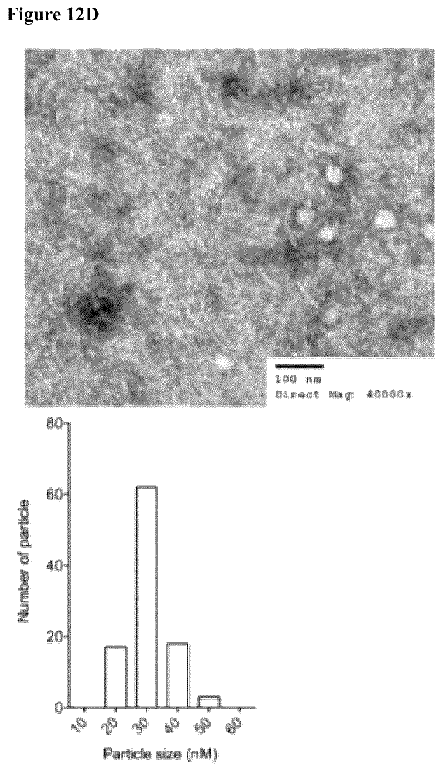

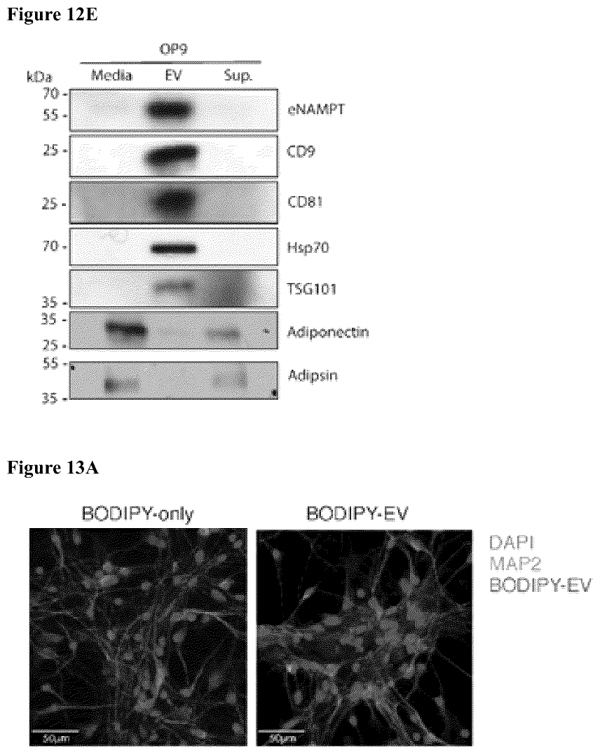

How circulating eNAMPT enhances tissue NAD+ biosynthesis has so far remained elusive. Our finding that eNAMPT enhances NAD+ biosynthesis in a tissue-specific manner suggested a possibility that circulating eNAMPT could directly contribute to NAD+ biosynthesis in its target tissues. In recent years, a transport mechanism of microRNA by EVs from one tissue to another has drawn much attention as an important mechanism of inter-tissue communications (Whitham et al., 2018; Ying et al., 2017; Zhang et al., 2017). Thus, we asked whether eNAMPT could also be transported by EVs in systemic circulation. We purified EVs from mouse plasma by conventional ultracentrifugation or by using a polymer-based total exosome isolation (TEI) kit. Whereas the yield of EVs from the TEI method was much higher than ultracentrifugation, both methods clearly showed that eNAMPT was highly enriched in the EV fraction, compared to whole plasma or the remaining non-EV fraction (FIG. 6A). The localization of eNAMPT in EVs was also confirmed by the enrichment of several EV markers including TG101, CD63, CD81, and CD9 and the depletion of transferrin and albumin. We also further purified the EV fractions from the TEI or ultracentrifugation by their floatation into a sucrose-density gradient. In both EV fractions, eNAMPT was clearly detected with several other EV markers, including Alix, TSG101, CD63, CD81, and CD9, in the third fraction from the sucrose-density gradient (FIGS. 6B and 12A). Additionally, we measured the densities of the fractions carrying eNAMPT-containing EVs isolated by the TEI method. eNAMPT was copurified with one of EV markers, Alix, in previously reported density fractions for EVs that range between 1.10 and 1.15 (FIG. 12B) (Ying et al., 2017). We also examined whether eNAMPT in human plasma was contained in EVs. Consistent with mouse eNAMPT, human plasma eNAMPT was mainly contained in the EV fraction (FIG. 6C). The proper enrichment of EVs from mouse and human plasma was further confirmed by electron microscopy (FIGS. 12C and 12D), which shows that the type of EVs purified from mouse and human plasma is consistent with the one characterized as small EVs (Durcin et al., 2017). Unfortunately, our attempt at immunogold labeling for EV-contained eNAMPT failed due to the unavailability of an appropriate antibody for this purpose. Thus, we examined whether eNAMPT in the plasma is protected from the protease treatment due to its localization within the EVs. Whereas the treatment of plasma with proteinase K nearly completely digested circulating plasma proteins such as transferrin and immunoglobulin light chain, eNAMPT and an EV marker TSG101 exhibited resistance to proteinase K digestion (FIG. 6D). Furthermore, upon adding a detergent (Triton-X) to dissolve the lipid bilayer of the EVs, the proteinase K treatment was able to eliminate eNAMPT and TSG101. These findings further confirmed that eNAMPT secreted into blood circulation was encapsulated into the EVs. By using cultured OP9 adipocytes, we also confirmed that fully differentiated adipocytes secrete EVs that contain eNAMPT and other EV marker proteins, but not other secreted proteins such as adiponectin and adipsin (FIG. 12E).

We found that the eNAMPT content in EVs dramatically decreased from 6 to 22 month-old mice (FIG. 6E). We also found that the content of eNAMPT in EVs from 24 month-old ANKI mice was significantly higher than that in age-matched control mice (FIG. 6F). These observed changes in EV-contained eNAMPT levels were physiologically important because proteomic comparisons of EV-contained proteins between young (6 month-old) and aged (24 month-old) mice and between aged ANKI and control mice demonstrated that only a small fraction (2-3%) of EV-contained proteins (181 proteins identified) exhibited significant changes in these conditions, and none of these proteins are related to NAD+ metabolism (FIG. 12F). Taken together, our results demonstrate that eNAMPT in mouse and human circulation is carried by EVs and also that changes in plasma eNAMPT levels observed during aging or in ANKI mice are due to the changes specific to EV-contained eNAMPT.

### Example 7: EV-Contained eNAMPT is Internalized into Cells and Directly Enhances NAD+ Biosynthesis

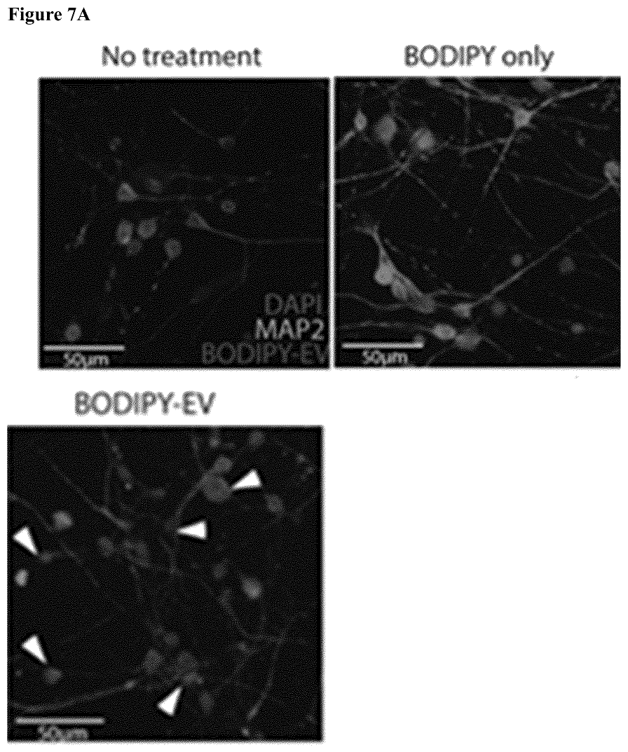

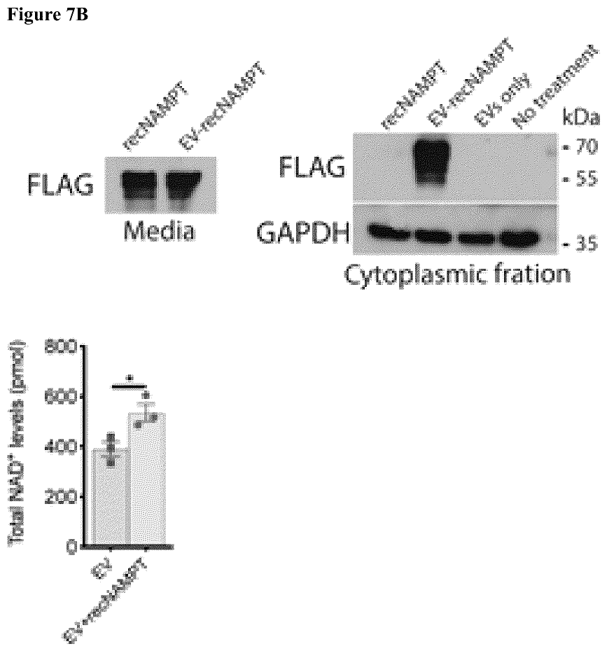

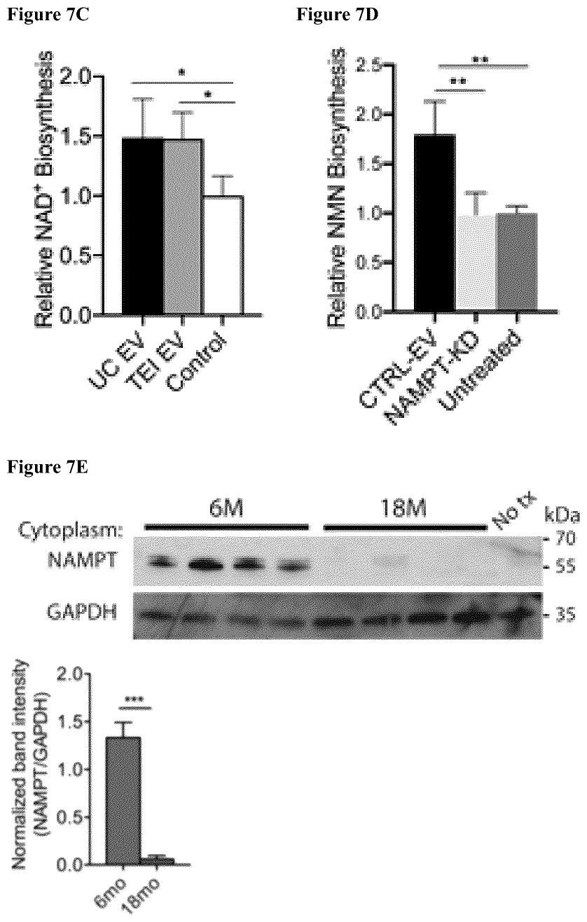

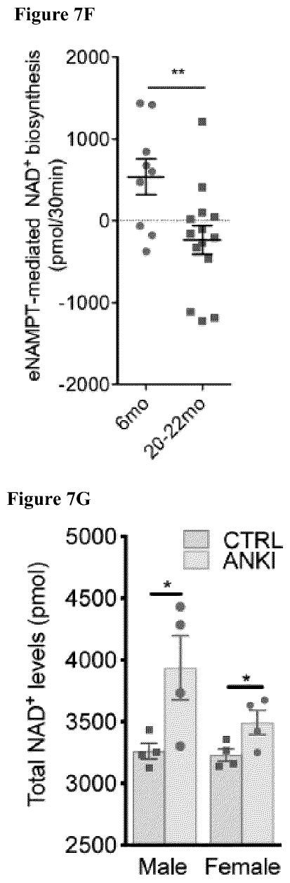

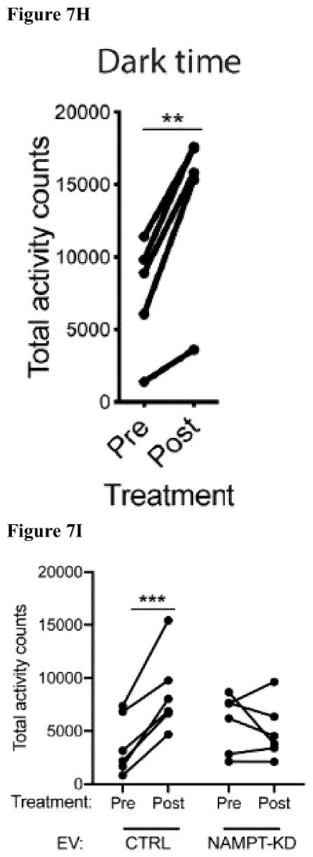

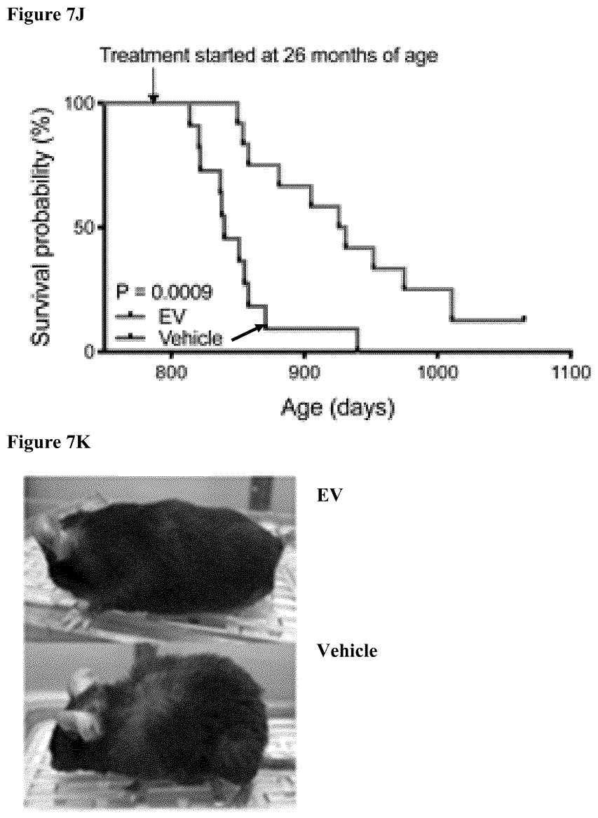

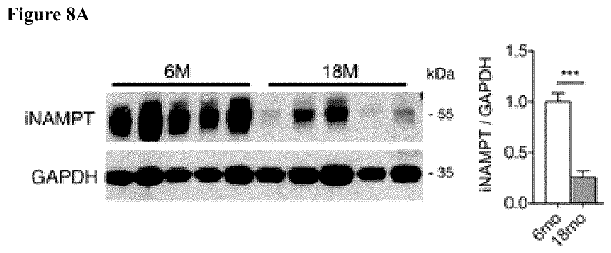

Having demonstrated eNAMPT localization within EVs, we next examined whether EV-contained eNAMPT could be internalized into cells and enhance NAD+ biosynthesis intracellularly. We first labeled isolated EVs with the BODIPY TR Ceramide, a red-fluorescent dye that can label lipid bilayers of EVs, and then incubated primary hypothalamic neurons with these BODIPY-labeled EVs. Primary hypothalamic neurons were labeled only when adding BODIPY-labeled EVs, but not when adding control BODIPY-treated media, suggesting that EVs were incorporated into primary hypothalamic neurons (FIG. 7A). FIG. 7. EV-contained eNAMPT directly enhances NAD+ biosynthesis in primary hypothalamic neurons and ameliorates age-associated decline in physical activity and extends lifespan in mice.

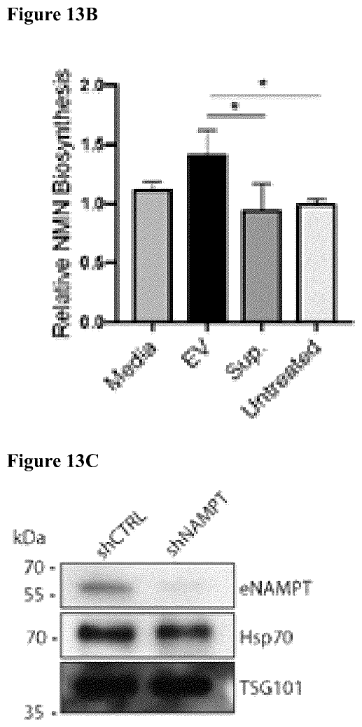

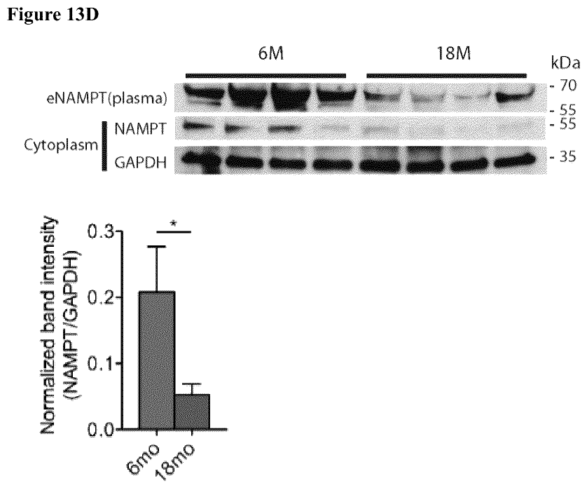

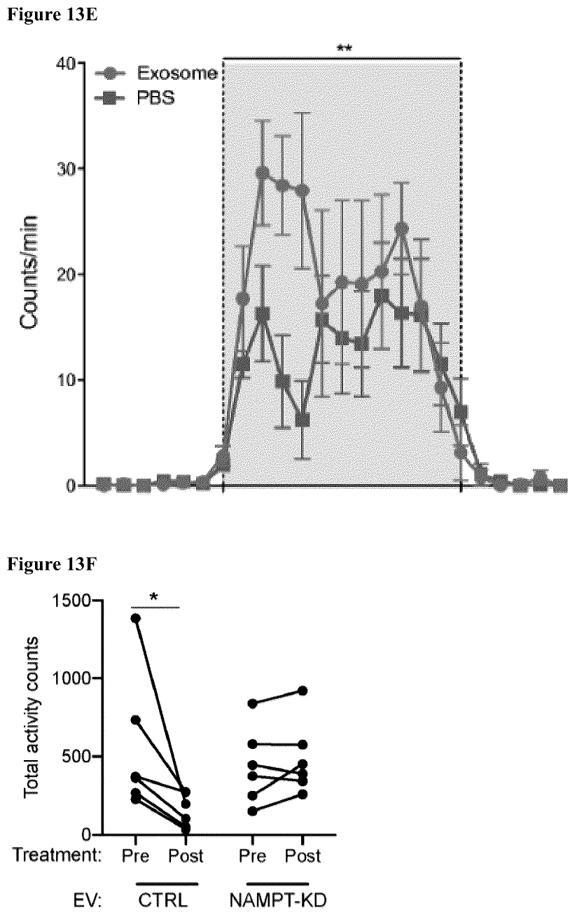

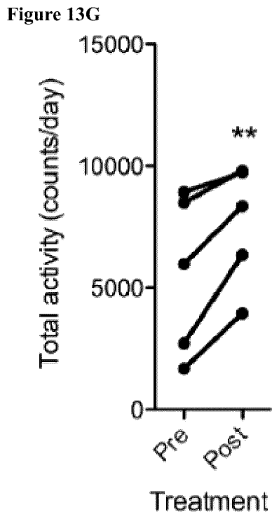

Next, we added bacterially produced FLAG-tagged recombinant NAMPT alone or FLAG-tagged recombinant NAMPT encapsulated into EVs to primary hypothalamic neurons. Interestingly, only EV-contained FLAG-tagged NAMPT was internalized into the cytoplasmic fraction of primary hypothalamic neurons (FIG. 7B). When incorporating FLAG-tagged recombinant NAMPT into purified EVs, these EVs with an additional amount of NAMPT were able to increase intracellular NAD+ levels in primary hypothalamic neurons (FIG. 7B, right panel). To further confirm the effects of EV-contained eNAMPT on cellular NAD+ biosynthesis, we measured the NAD+ biosynthetic rate using isotopically labeled nicotinamide (D-4-NAM) in primary hypothalamic neurons. Both EVs (1 μg/μl as protein concentration) purified from mouse plasma by ultracentrifugation and the TEI method showed equivalent increases in NAD+ biosynthesis compared to controls (FIG. 7C). To examine whether the observed effects on NAD+ biosynthesis are due to the enzymatic activity of EV-contained eNAMPT, we used EVs purified from the culture media of OP9 adipocytes by ultracentrifugation. We confirmed that ultracentrifugation-purified EVs from OP9 adipocytes were internalized properly to primary hypothalamic neurons (FIG. 13A). Using this OP9 adipocyte system, we demonstrated that concentrated EVs from OP9 culture media, but not the EV-depleted supernatant, showed significant enhancement of NMN biosynthesis in primary hypothalamic neurons (FIG. 13B). Then we compared the effects of EVs purified from the media of control and Nampt-knockdown (Nampt-KD) OP9 adipocytes on the NMN biosynthesis in primary hypothalamic neurons. The NAMPT expression was reduced by 80% in EVs secreted from Nampt-KD OP9 adipocytes (FIG. 13C). Whereas EVs purified from the culture media of control OP9 adipocytes clearly showed the enhancement of NMN biosynthesis, EVs purified from the culture media of Nampt-KD OP9 adipocytes showed no enhancement of NMN biosynthesis in primary hypothalamic neurons (FIG. 7D), demonstrating that these effects of EVs to stimulate cellular NMN/NAD+ biosynthesis is primarily due to EV-contained eNAMPT.

The internalization of EV-contained eNAMPT into the cytoplasm of primary hypothalamic neurons was also examined by using mouse plasma and purified EVs from 6 and 18 month-old mice (FIGS. 13D and 7E). The amounts of internalized NAMPT in the cytoplasm mirrored the amounts of eNAMPT contained in original plasma or purified EVs, indicating that EVs from young mice can deliver higher amounts of eNAMPT into cells, compared to those from aged mice. We next compared the capabilities of EVs purified from young and aged mouse plasma to stimulate NAD+ biosynthesis in primary hypothalamic neurons. Increments in intracellular NAD+ levels were significantly higher with EVs from 6 month-old mice compared to those from 20-22 month-old mice (FIG. 7F). Furthermore, EVs from ANKI male and female mice were able to increase intracellular NAD+ levels in primary hypothalamic neurons, compared to those from age-matched control mice (FIG. 7G). These results provide compelling evidence that EV-contained eNAMPT, which is internalized into target cells, can contribute to the enhancement of NMN/NAD+ biosynthesis within its target cells.

### Example 8: Supplementation with EV-Contained eNAMPT Enhances Wheel-Running Activity and Extends Lifespan in Aged Mice

Given that EV-contained eNAMPT was able to enhance intracellular NAD+ levels in primary hypothalamic neurons, we reasoned that supplementation with eNAMPT-containing EVs could convey similar anti-aging effects on aged wild-type mice, as observed in aged ANKI mice. To test this possibility, we injected EVs purified from the plasma of 4-6 month-old mice intraperitoneally into 20 month-old wild-type female mice for four consecutive days. Remarkably, supplementation with EVs purified from young mouse plasma significantly enhanced wheel-running activity in aged mice during the dark time, compared to PBS-injected age-matched control mice (FIGS. 13E and 7H). Interestingly, whereas the wheel-running activity during the dark time clearly increased, the activity during the light time decreased significantly, implying that those EV-injected aged mice may sleep better. To further confirm whether this effect is due to EV-contained eNAMPT, we compared wheel-running activities of aged wild-type female mice by injecting EVs purified from the culture media of control and Nampt-KD OP9 adipocytes. Whereas EVs purified from control OP9 culture media again exhibited significant increases and decreases in wheel-running activity during the dark and the light times, respectively, EVs purified from Nampt-KD OP9 culture media failed to show these effects (FIGS. 7I and 13F), demonstrating that this effect is mediated by EV-contained eNAMPT. A mild enhancement of wheel-running activity was also observed in aged male mice (FIG. 13G), suggesting that male mice can also respond to high levels of EV-contained eNAMPT supplementation.

We then tested whether eNAMPT-containing EVs purified from young mouse plasma could extend the lifespan of aged mice. We started injecting EVs purified from young-to-middle age (4-12 month-old) mice once a week into female mice at 26 months of age. Remarkably, supplementation with EVs purified from young-to-middle age mice significantly extended the lifespan of aged mice (FIG. 7J). The median lifespan was extended by 10.2% (840 days for vehicle-injected mice versus 926 days for EV-injected mice, Gehan-Breslow-Wilcoxon test, ²=11.10, df=1, p=0.0009), and two mice were still alive at the time results were recorded. The mean maximal lifespans of 4 longest-lived mice in each group were 881±63.6 and 999±47 days for vehicle- and EV-injected mice, respectively (Student's t test, p=0.0016, 13.3% extension). The EV-injected aged mice generally maintained much healthier looking and higher activity compared to vehicle-injected age-matched control mice (FIG. 7K). Taken together, these findings demonstrate that supplementation with EV-contained eNAMPT is an effective anti-aging intervention to mitigate age-associated functional decline and extend lifespan in mice.

The results in Examples 1 to 8 demonstrates the importance of a novel EV-mediated inter-tissue communication mechanism that delivers eNAMPT, a key NAD+ biosynthetic enzyme, to specific tissues in controlling the process of aging and determining healthspan and lifespan in mice. Age-associated decline in the levels of circulating EV-contained eNAMPT limits NAD+ availability and tissue functions in these target tissues, including the hypothalamus, hippocampus, pancreas, and retina. Further it was shown that supplementing EV-contained eNAMPT to aged mice genetically or pharmacologically mitigates age-associated physiological decline during aging and extends lifespan in mice.

Because the hypothalamus has been suggested to function as a high-order control center of aging in mammals (Satoh et al., 2013; Zhang et al., 2013; Zhang et al., 2017), it was hypothesized that eNAMPT secreted from adipose tissue plays a critical role in affecting the process of aging and eventually lifespan. To address this hypothesis, we generated adipose tissue-specific Nampt knock-in (ANKI) mice (Yoon et al., 2015) and characterized their aging phenotypes. Interestingly, aged ANKI mice maintained youthful levels of circulating eNAMPT and increased NAD+ levels in multiple tissues including the hypothalamus, hippocampus, pancreas, and retina, exhibiting significant improvement in physical activity, sleep quality, cognitive function, glucose metabolism, and photoreceptor functions. With these beneficial effects against aging, ANKI mice, particularly females, showed a significant extension of healthspan. Surprisingly, we found that eNAMPT was carried in extracellular vesicles (EVs) through blood circulation in mice and humans. EV-contained eNAMPT was internalized into primary hypothalamic neurons and enhanced NAD+ biosynthesis intracellularly. Injecting eNAMPT-containing EVs purified from young mice or cultured adipocytes, but not from Nampt-knockdown adipocytes, was able to enhance wheel-running activity and extend lifespan in aged mice. These findings demonstrate a novel inter-tissue communication mechanism driven by an EV-mediated delivery of eNAMPT. This new physiological system mediated by EV-contained eNAMPT plays a critical role in maintaining systemic NAD+ biosynthesis and counteracting age-associated physiological decline, implicating EV-contained eNAMPT as a potential anti-aging biologic in humans.

Examples 1 to 8 herein demonstrate that EV-mediated systemic delivery of eNAMPT mitigates age-associated functional decline in specific target tissues including the hypothalamus, hippocampus, pancreas, and retina, delays age-associated mortality rate, and extends healthspan and lifespan in mice. The surprising finding in this study is that EV-contained eNAMPT is internalized into target cells and enhances NMN/NAD+ biosynthesis intracellularly, whereas the NAMPT protein alone cannot be internalized by itself. This provides a critical resolution for a long-standing debate on the physiological importance and function of eNAMPT in mammals. Whereas eNAMPT can function as a systemic NAD+ biosynthetic enzyme and enhance NAD+, SIRT1 activity, and neural activation in the hypothalamus (Revollo et al., 2007; Yoon et al., 2015), eNAMPT has also been reported to function as a proinflammatory cytokine (Dahl et al., 2012). Given that eNAMPT in circulation is almost exclusively contained in EVs under physiological conditions and also that only EV-contained eNAMPT is properly internalized into the cytoplasmic fraction of cells, we suggest that the physiological relevance and function of eNAMPT is to maintain NMN/NAD+ biosynthesis systemically, particularly in the tissues that have relatively low levels of iNAMPT, such as the hypothalamus, hippocampus, pancreas, and retina. Although the precise mechanism by which eNAMPT-containing EVs are targeted specifically to those tissues needs to be elucidated, this EV-mediated systemic delivery of eNAMPT is a novel inter-tissue communication mechanism that maintains NAD+ homeostasis throughout the body and modulates the process of aging and lifespan in mammals.

### Systemic Decline in NAMPT-Mediated NAD+ Biosynthesis Limits Tissue Functions During Aging

In mammals, NAMPT is the rate-limiting enzyme in a major NAD+ biosynthetic pathway starting from nicotinamide, a form of vitamin B3. It has now been well established that systemic NAD+ availability declines dramatically over age, and that age-associated reduction in iNAMPT levels contributes to limiting NAD+ availability in many tissues (Yoshino et al., 2018). We now show that circulating eNAMPT levels also decline with age in mice and humans, limiting NAD+ availability in specific tissues that rely on eNAMPT-mediated NAD+ biosynthesis. Adipose tissue-specific overexpression of Nampt maintains circulating eNAMPT levels, resulting in significant enhancement in physical activity, sleep quality, glucose-stimulated insulin secretion, retinal photoreceptor function, and cognitive function in aged mice. These remarkable anti-aging effects of eNAMPT also contribute to the extension of median lifespan in mice. In aged ANKI mice, we did not observe any significant adverse effects of eNAMPT, including inflammation and cancer risks, arguing against the proposed primary function of eNAMPT as a proinflammatory cytokine.

Because circulating eNAMPT levels decline with age, an individual's capacity to sustain high levels of circulating eNAMPT must be important to maintain functional homeostasis of tissues over time, which likely determines a healthspan of each individual. The results from our small prospective study provides compelling support for this notion, showing a significant correlation between circulating eNAMPT levels and the remaining lifespan. Given that eNAMPT secretion from adipose tissue is regulated in a NAD+/SIRT1-dependent manner (Yoon et al., 2015), the reservoir and/or the turnover of adipose NAD+ could be a critical determinant for circulating eNAMPT levels and thereby lifespan. Interestingly, it has been reported that the effect of lifespan extension by diet restriction correlates inversely with fat reduction measured at mid-life and later ages, suggesting that certain factors associated with fat are important for survival and lifespan extension under diet restriction (Liao et al., 2011). Based on our results, it would be of great interest to examine whether eNAMPT secreted from adipose tissue is a significant contributor to the delayed aging and lifespan-extending effects of diet restriction.

Genetic Supplementation of eNAMPT Delays Aging and Extends Healthspan in Mice

Aged ANKI mice show remarkable enhancement of NAD+ levels and tissue functions in the hypothalamus, hippocampus, pancreas, and retina. We have previously demonstrated that NAMPT-mediated NAD+ biosynthesis and NAD+-dependent sirtuins play important roles in regulating these tissue functions. In the hypothalamus, NAD+/SIRT1 signaling is critical in controlling the process of aging and determining lifespan (Satoh et al., 2013). In the hippocampus, NAMPT plays an important role in the function of excitatory neurons (Stein et al., 2014), particularly neurons in the CA1 region (Johnson et al., 2018). In pancreatic β cells, NAMPT and SIRT1 are critical to regulate glucose-stimulated insulin secretion (Moynihan et al., 2005; Revollo et al., 2007). In the retina, NAMPT and mitochondrial sirtuins SIRT3/5 are essential for the function of rod and cone photoreceptor neurons (Lin et al., 2016; Mills et al., 2016). These tissues most likely represent a group of tissues that are the most vulnerable to NAD+ decline. There may be other tissues to which eNAMPT is also targeted to maintain adequate NAD+ biosynthesis. Considering that adipose tissue is a major source of circulating eNAMPT (Yoon et al., 2015), it will be important to further elucidate inter-tissue communications between adipose tissue and other tissues through EV-mediated eNAMPT delivery.

Interestingly, phenotypes of aged ANKI mice overlap with those of aged BRASTO mice (Satoh et al., 2013). Particularly, the enhancement of wheel-running activity and sleep quality are observed in both aged ANKI and BRASTO mice. Consistent with these phenotypes, the hypothalamic expression levels of Ox2r and Prdm13, two SIRT1 target genes responsible for those phenotypes (Satoh et al., 2013; Satoh et al., 2015), are significantly increased in both mouse models. Nonetheless, whereas BRASTO mice exhibit both median and maximal lifespan extension, ANKI mice show only median lifespan extension. This discrepancy between BRASTO and ANKI mice suggests an interesting possibility that the level of SIRT1 in hypothalamic neurons primarily determines a maximal level of their function and thereby limits maximal lifespan, whereas the level of circulating eNAMPT modulates the extent of hypothalamic neuronal function and thereby changes median lifespan accordingly. Given that continuous supplementation with eNAMPT-containing EVs extends median and maximal lifespan of aged mice, it is also possible that the effect of eNAMPT in ANKI mice might be hindered at a very late stage of aging by a reduction in adipose tissue mass.

### Example 9

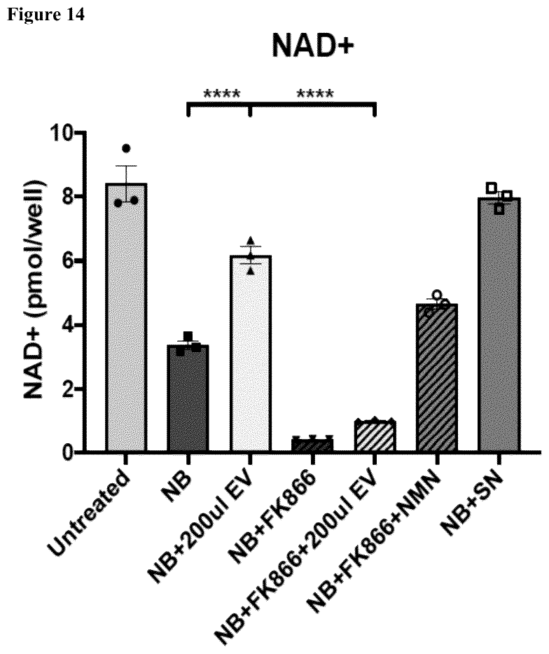

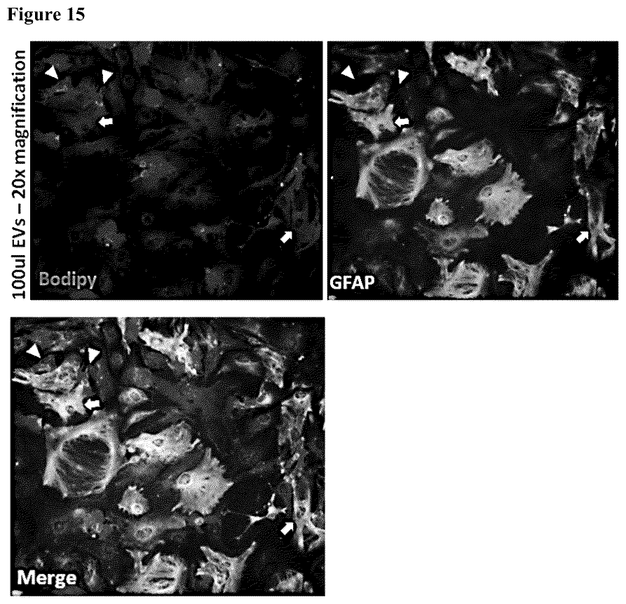

Primary neurons were isolated from mouse embryos at E16 and treated after 7 days in vitro with neurobasal medium (NB) containing the denoted combinations of the following additives: Untreated-neurons were left in standard neurobasal culture medium, which includes B27, N2, and glutamine supplements; NB—neurobasal without additives; 200 μl EV-EVs extracted from 200 μl of mouse plasma, FK866 (10 nM)—NAMPT inhibitor; NMN—250 μM of nicotinamide mononucleotide (NAD+ precursor); SN—supernatant serum recovered from plasma after EV extraction, combined with NB at a ½ ratio. NAD+ levels were measured after 30 minutes of applicable treatment through NAD/NADH-Glo fluorescent kit. FIG. 14 illustrates the induction of NAD+ biosynthesis in cultured primary mouse hippocampal neurons through treatment of extracellular vesicle (EV)-contained eNAMPT. NAD+ levels decreased in neurons cultured in nutrient depleted, NB only, media, an effect partially rescued by supplementation with EVs. These data show that EVs are sufficient to increase neuronal NAD+ content. This increase is blocked with FK866 treatment, suggesting that the increased NAD+ induced by EV treatment is dependent on NAMPT.

Primary hippocampal glial cultures were isolated from p2 pups and shaken to remove less-adherent microglia. EVs isolated from mouse plasma were labeled with the sphingolipid dye Bodipy-TR-Ceramide. Astrocyte-enriched cultures are treated with these labeled EVs for 30 mins, followed by fixation and immunofluorescent staining for the astrocyte marker GFAP. FIG. 15 shows low levels of EV uptake in primary mouse GFAP+ astrocytes and more substantial EV uptake in GFAP-cells. Low levels of Bodipy dye can be observed in GFAP+ astrocytes (white arrows) indicating some uptake of EVs by astrocytes. However, more substantial staining is clearly visible in GFAP-cells, some of which display obvious microglial morphology (white arrowheads). These data show that while astrocytes are capable of taking up EVs, the affinity of astrocytes for EVs may be lower than that of other cell types.

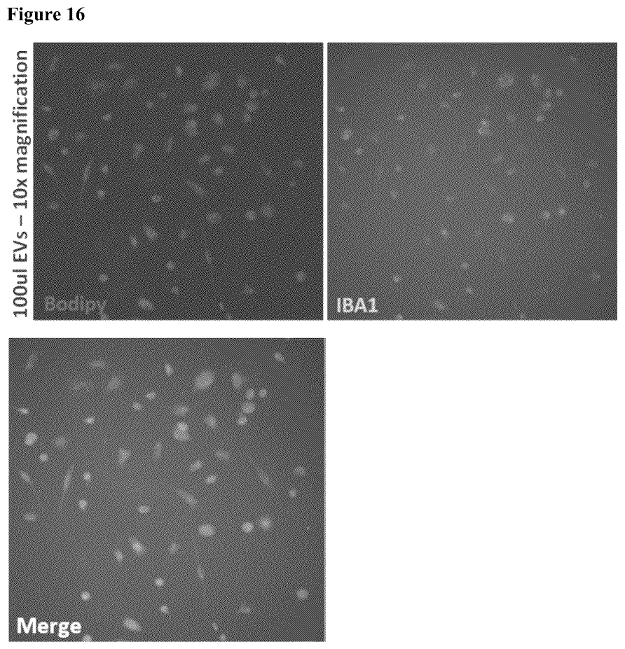

FIG. 16 demonstrates marked uptake of EVs by microglia. Primary mouse hippocampal microglial cells shaken off of astrocyte-enriched cultures (see FIG. 15) were replated and similarly treated with Bodipy-labeled EVs. Consistent colocalization of Bodipy signal with immunofluorescent labeling for the microglial marker IBA1 indicates considerable uptake of EVs by primary microglia. These data suggest that microglia, at least in culture, have a high-affinity for EV uptake.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained. As various changes could be made in the above methods, processes, and compositions without departing from the scope of the invention, it is intended that all matter contained in the above description and shown in the accompanying drawings shall be interpreted as illustrative and not in a limiting sense.

1.-37. (canceled)

38. A method of increasing NMN and/or NAD+ biosynthesis in a subject, the method comprising administering to the subject a composition comprising extracellular nicotinamide phosphoribosyltransferase (eNAMPT) and/or mutant thereof and an extracellular vesicle.

39. A method of preventing or treating an age-associated condition in a subject, the method comprising administering to the subject a composition comprising extracellular nicotinamide phosphoribosyltransferase (eNAMPT) and/or mutant thereof and an extracellular vesicle.

40. The method of claim 39, wherein the age-associated condition comprises a physiological condition selected from the group consisting of a decline in physical activity, decline in sleep quality, decline in cognitive function, decline in glucose metabolism, decline in vision, and combinations thereof.

41. The method of claim 39, wherein the composition is administered parenterally.

42. The method of claim 39, wherein the subject is a human.

43. The method of claim 42, wherein from about 10 to about 500 mg of eNAMPT and/or mutant thereof is administered per day to the subject.

44. A method of increasing NMN and/or NAD+ biosynthesis in a cell, the method comprising applying a composition comprising extracellular nicotinamide phosphoribosyltransferase (eNAMPT) and/or mutant thereof and an extracellular vesicle to the cell.

45. A process for preparing a composition comprising extracellular nicotinamide phosphoribosyltransferase (eNAMPT) and/or mutant thereof and an extracellular vesicle, the process comprising:
subjecting a medium comprising vesicles comprising the extracellular vesicle and eNAMPT and/or mutant thereof to a separation process to obtain an enriched vesicle fraction, wherein the concentration of the vesicles in the enriched vesicle fraction is greater than the concentration of the vesicles in the medium.

46. The process of claim 45, wherein the medium is selected from the group consisting of a culture comprising adipocytes, blood, and blood plasma.

47. The process of claim 45, wherein the medium comprises a culture comprising adipocytes.

48. The process of claim 47, wherein the adipocytes overexpress a gene that codes for eNAMPT and/or mutant thereof.

49. The process of claim 47, wherein the medium comprises blood or blood plasma.

50. The process of claim 45, wherein the separation process comprises centrifugation.

51. The process of claim 50, wherein the separation process comprises ultracentrifugation.

52. The process of claim 45, wherein the separation process comprises an exosome isolation technique.

53. The process of claim 45, further comprising mixing the enriched vesicle fraction or fraction derived therefrom with a carrier.

54. (canceled)

55. The method of claim 38, wherein the mutant of eNAMPT comprises at least 99% sequence identity to the wild-type NAMPT of SEQ ID NO: 1 or SEQ ID NO: 2 and further comprises an amino acid sequence having an arginine residue at a position corresponding to position 53 of wild-type NAMPT of SEQ ID NO: 1 or an amino acid sequence having an arginine residue at a position corresponding to position 53 of wild-type NAMPT of SEQ ID NO: 2.

56. The method of claim 39, wherein the mutant of eNAMPT comprises at least 99% sequence identity to the wild-type NAMPT of SEQ ID NO: 1 or SEQ ID NO: 2 and further comprises an amino acid sequence having an arginine residue at a position corresponding to position 53 of wild-type NAMPT of SEQ ID NO: 1 or an amino acid sequence having an arginine residue at a position corresponding to position 53 of wild-type NAMPT of SEQ ID NO: 2.

57. The method of claim 44, wherein the mutant of eNAMPT comprises at least 99% sequence identity to the wild-type NAMPT of SEQ ID NO: 1 or SEQ ID NO: 2 and further comprises an amino acid sequence having an arginine residue at a position corresponding to position 53 of wild-type NAMPT of SEQ ID NO: 1 or an amino acid sequence having an arginine residue at a position corresponding to position 53 of wild-type NAMPT of SEQ ID NO: 2.

58. The method of claim 45, wherein the mutant of eNAMPT comprises at least 99% sequence identity to the wild-type NAMPT of SEQ ID NO: 1 or SEQ ID NO: 2 and further comprises an amino acid sequence having an arginine residue at a position corresponding to position 53 of wild-type NAMPT of SEQ ID NO: 1 or an amino acid sequence having an arginine residue at a position corresponding to position 53 of wild-type NAMPT of SEQ ID NO: 2.


SEQUENCE LISTING (6 sequences)

SEQ ID NO: 1 - protein, 491 aa, Mus musculus
MNAAAEAEFNILLATDSYKVTHYKQYPPNTSKVYSYFECREKKTENSKVRKVKYEETVFYGLQYILNKYLKGKVVTKEKIQEAKEVYREHFQDDVFNERGWNYILEKYDGHLPIEVKAVPEGSVIPRGNVLFTVENTDPECYWLTNWIETILVQSWYPITVATNSREQKKILAKYLLETSGNLDGLEYKLHDFGYRGVSSQETAGIGASAHLVNFKGTDTVAGIALIKKYYGTKDPVPGYSVPAAEHSTITAWGKDHEKDAFEHIVTQFSSVPVSVVSDSYDIYNACEKIWGEDLRHLIVSRSTEAPLIIRPDSGNPLDTVLKVLDILGKKFPVTENSKGYKLLPPYLRVIQGDGVDINTLQEIVEGMKQKKWSIENVSFGSGGALLQKLTRDLLNCSFKCSYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 2 - protein, 491 aa, Homo sapiens [sequence omitted]

SEQ ID NO: 3 - protein, 491 aa, Mus musculus
MNAAAEAEFNILLATDSYKVTHYKQYPPNTSKVYSYFECREKKTENSKVRKVRYEETVFYGLQYILNKYLKGKVVTKEKIQEAKEVYREHFQDDVFNERGWNYILEKYDGHLPIEVKAVPEGSVIPRGNVLFTVENTDPECYWLTNWIETILVQSWYPITVATNSREQKKILAKYLLETSGNLDGLEYKLHDFGYRGVSSQETAGIGASAHLVNFKGTDTVAGIALIKKYYGTKDPVPGYSVPAAEHSTITAWGKDHEKDAFEHIVTQFSSVPVSVVSDSYDIYNACEKIWGEDLRHLIVSRSTEAPLIIRPDSGNPLDTVLKVLDILGKKFPVTENSKGYKLLPPYLRVIQGDGVDINTLQEIVEGMKQKKWSIENVSFGSGGALLQKLTRDLLNCSFKCSYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 4 - protein, 491 aa, Homo sapiens
MNPAAEAEFNILLATDSYKVTHYKQYPPNTSKVYSYFECREKKTENSKLRKVRYEETVFYGLQYILNKYLKGKVVTKEKIQEAKDVYKEHFQDDVFNEKGWNYILEKYDGHLPIEIKAVPEGFVIPRGNVLFTVENTDPECYWLTNWIETILVQSWYPITVATNSREQKKILAKYLLETSGNLDGLEYKLHDFGYRGVSSQETAGIGASAHLVNFKGTDTVAGLALIKKYYGTKDPVPGYSVPAAEHSTITAWGKDHEKDAFEHIVTQFSSVPVSVVSDSYDIYNACEKIWGEDLRHLIVSRSTQAPLIIRPDSGNPLDTVLKVLEILGKKFPVTENSKGYKLLPPYLRVIQGDGVDINTLQEIVEGMKQKMWSIENIAFGSGGGLLQKLTRDLLNCSFKCSYVVTNGLGINVFKDPVADPNKRSKKGRLSLHRTPAGNFVTLEEGKGDLEEYGQDLLHTVFKNGKVTKSYSFDEIRKNAQLNIELEAAHH

SEQ ID NO: 5 - protein, 491 aa, Mus musculus
MNAAAEAEFNILLATDSYKVTHYKQYPPNTSKVYSYFECREKKTENSKVRKVQYEETVFYGLQYILNKYLKGKVVTKEKIQEAKEVYREHFQDDVFNERGWNYILEKYDGHLPIEVKAVPEGSVIPRGNVLFTVENTDPECYWLTNWIETILVQSWYPITVATNSREQKKILAKYLLETSGNLDGLEYKLHDFGYRGVSSQETAGIGASAHLVNFKGTDTVAGIALIKKYYGTKDPVPGYSVPAAEHSTITAWGKDHEKDAFEHIVTQFSSVPVSVVSDSYDIYNACEKIWGEDLRHLIVSRSTEAPLIIRPDSGNPLDTVLKVLDILGKKFPVTENSKGYKLLPPYLRVIQGDGVDINTLQEIVEGMKQKKWSIENVSFGSGGALLQKLTRDLLNCSFKCSYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 6 - protein, 491 aa, Homo sapiens
MNPAAEAEFNILLATDSYKVTHYKQYPPNTSKVYSYFECREKKTENSKLRKVQYEETVFYGLQYILNKYLKGKVVTKEKIQEAKDVYKEHFQDDVFNEKGWNYILEKYDGHLPIEIKAVPEGFVIPRGNVLFTVENTDPECYWLTNWIETILVQSWYPITVATNSREQKKILAKYLLETSGNLDGLEYKLHDFGYRGVSSQETAGIGASAHLVNFKGTDTVAGLALIKKYYGTKDPVPGYSVPAAEHSTITAWGKDHEKDAFEHIVTQFSSVPVSVVSDSYDIYNACEKIWGEDLRHLIVSRSTQAPLIIRPDSGNPLDTVLKVLEILGKKFPVTENSKGYKLLPPYLRVIQGDGVDINTLQEIVEGMKQKMWSIENIAFGSGGGLLQKLTRDLLNCSFKCSYVVTNGLGINVFKDPVADPNKRSKKGRLSLHRTPAGNFVTLEEGKGDLEEYGQDLLHTVFKNGKVTKSYSFDEIRKNAQLNIELEAAHH